(19) United States
(12) Patent Application Publication
(10) Pub. No.: US 2008/0213187 A1
(43) Pub. Date: Sep. 4, 2008

(54) NOVEL CASCADE POLYMER COMPLEXES, PROCESSES FOR THEIR PREPARATION AND PHARMACEUTICAL COMPOSITIONS COMPRISING THEM

(76) Inventors: Heribert SCHMITT-WILLICH, Berlin (DE); Heiko Schirmer, Berlin (DE); Bernd Misselwitz, Glienicke (DE); Hanns-Joachim Weinmann, Berlin (DE); Peter Caravan, Cambridge, MA (US)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 12/015,769

(22) Filed: Jan. 17, 2008

Related U.S. Application Data
(60) Provisional application No. 60/885,497, filed on Jan. 18, 2007.

(30) Foreign Application Priority Data
Jan. 18, 2007 (DE) 10 2007 002 726.7

(51) Int. Cl. A61K 49/06; C07C 69/76; C07C 39/04

(52) U.S. Cl. 424/9.3; 568/747; 560/76

(57) ABSTRACT

The invention relates to novel cascade polymer complexes, to compositions comprising these compounds, to the use of the complexes in NMR diagnosis, and to processes for preparing these compounds and compositions. The complex-forming cascade polymer complexes according to the invention can be described by general formula I:

R-L-A-{X—[Y-(Z-{W—K_{w}}_{z})_{y}]ₓ}ₐ₋₁ (I)
where
R=is an HSA-binding unit,
L=is a linker or a bond,
A=is a nitrogen-containing cascade core of base multiplicity a,
X and Y are independently of one another a direct linkage or a cascade reproduction unit of reproduction multiplicity x and y, respectively,
Z and W=are independently of one another a direct linkage or a cascade reproduction unit of reproduction multiplicity z and w, respectively,
K is the residue of a complexing agent,
a=is numbers 2 to 12, and
x, y, z and w are independently of one another the numbers 1 to 4,
with the proviso that exactly one multiplicity of the base multiplicity a of the cascade core A represents exactly one point of linkage to L, and
with the proviso that the cascade polymer complexes comprise in the complexing agent residue K in total at least 4 ions of an element of atomic number 20 to 29, 39, 42 to 44 or 57 to 83 and comprise where appropriate cations of inorganic and/or organic bases, amino acids or amino amides.

This application claims the benefit of the filing date of U.S. Provisional Application Ser. No. 60/885,497 filed Jan. 18, 2007.

The present invention relates to the subject-matter characterized in the claims, that is to say novel cascade polymer complexes, compositions comprising these compounds, the use of the complexes in diagnosis, and processes for preparing these compounds and compositions.

The contrast media employed clinically at present for the modern imaging method of magnetic resonance imaging (MRI or NMR) [e.g. Magnevist®, or ProHance®] are distributed in the whole extracellular space of the body (intravascular and interstitium). This distribution space comprises about 20% of the body's volume.

Extracellular MRI contrast media were first employed successfully for the clinical diagnosis of cerebral and spinal disease processes because the situation here with regard to the regional distribution space is quite special. In the brain and in the spinal cord, extracellular contrast media in healthy tissue cannot leave the intravascular space owing to the blood-brain barrier. In cases of disease processes with impairment of the blood-brain barrier (e.g. malignant tumours, inflammations, demyelinizing disorders etc.), regions with increased blood vessel permeability for these extracellular contrast media then arise within the brain (Schmiedl et al., MRI of blood-brain barrier permeability in astrocytic gliomas: application of small and large molecular weight contrast media, Magn. Reson. Med. 22: 288, 1991). It is possible by exploiting this impairment of vascular permeability to recognize diseased tissue with high contrast compared with the healthy tissue.

Outside the brain and the spinal cord, however, there is no such permeability barrier for the abovementioned contrast media (Canty et al., First-pass entry of nonionic contrast agent into the myocardial extravascular space. Effects on radiographic estimate of transit time and blood volume. Circulation 84: 2071, 1991). Hence, accumulation of the contrast medium is no longer dependent on vascular permeability but now only on the size of the extracellular space in the corresponding tissue. It is not possible to distinguish the vessels from the surrounding interstitial space on use of these contrast media.

A contrast medium which is distributed exclusively in the vascular space would be desirable in particular for the visualization of vessels. Such a blood-pool agent should make it possible to distinguish well perfused from poorly perfused tissue by means of magnetic resonance imaging and thus to diagnose ischaemia. It would also be possible to distinguish infarcted tissue owing to its anaemia from surrounding healthy or ischaemic tissue on using a vascular contrast medium. This is of particular importance when the objective is for example to distinguish a myocardial infarction from an ischaemia.

To date, most patients suspected of a cardiovascular disorder (this disorder is the commonest cause of death in Western industrialized countries) have had to undergo invasive diagnostic investigations. In angiography at present in particular X-ray diagnosis with the aid of iodine-containing contrast media is used. These investigations are associated with various disadvantages: they are linked to the risk of radiation stress and with inconveniences and stresses arising in particular from the fact that iodine-containing contrast media must be used in very much higher concentration compared with NMR contrast media.

Hence there is a need for NMR contrast media which can mark the vascular space (blood-pool-agent). These compounds should be notable for good tolerability and for a high activity (large increase in the signal intensity in MRI).

The approach to solving at least part of these problems by using complexing agents linked to macromolecules or biomolecules has to date had only very limited success.

Thus, for example, the number of paramagnetic centres in the complexes described in European patent applications No. 0 088 695 and No. 0 150 844 is insufficient for satisfactory imaging.

Increasing the number of necessary metal ions by multiple introduction of complexing units into a macromolecular biomolecule is associated with a non-tolerable impairment of the affinity and/or specificity of this biomolecule [J. Nucl. Med. 24, 1158 (1983)].

Macromolecules may generally be suitable as contrast media for angiography. However, albumin-GdDTPA (Radiology 1987; 162: 205) for example shows 24 hours after intravenous injection in rats an accumulation in liver tissue which is almost 30% of the dose. In addition, only 20% of the dose is eliminated in 24 hours.

The macromolecule polylysine-GdDTPA (European patent application, publication No. 0 233 619) has likewise proved suitable as blood-pool agent. However, this compound consists owing to its preparation of a mixture of molecules of varying size. It was possible to show in excretion tests in rats that this macromolecule is excreted unchanged by glomerular filtration via the kidney. However, owing to the synthesis, polylysine-GdDTPA may also contain macromolecules which are so large that they cannot pass through the capillaries of the kidney in glomerular filtration and thus remain in the body.

Macromolecular contrast media based on carbohydrate, e.g. dextran, have also been described (European patent application, publication No. 0 326 226). The disadvantage of these compounds is that as a rule they carry only about 5% of the signal-enhancing paramagnetic cation.

The polymers described in European patent publication No. 0 430 863 represent an advance along the path to blood-pool agents because they no longer have the heterogeneity in size and molecular mass characteristic of the polymers previously mentioned. However, they are still unsatisfactory in relation to a prolonged residence time in the blood, complete excretion, tolerability and/or activity.

A further advance along the path to MR blood-pool agents is represented by European patent EP 0 836 485 (Schmitt-Willich et al.) because it describes polymers which are well tolerated and completely excretable. However, especially in the use as coronary angiography contrast media and on use of higher magnetic fields such as, for example 1.5 or 3 Tesla, a higher concentration and, where appropriate, a longer residence time of the gadolinium complexes in the blood is furthermore desirable.

The object therefore was to provide novel diagnostic means in particular for the identification and localization of vascular disorders which do not have the disadvantages mentioned. This object is achieved by the present invention.

It has been found that complexes which comprise a nitrogen-containing cascade polymer provided with complex-forming ligands, at least 4 ions of an element of atomic numbers 20-29, 39, 42, 44 or 57-83, an HSA binding unit, where appropriate a linker, and where appropriate cations of inorganic and/or organic bases, amino acids or amino amides, and comprise where appropriate acylated amino groups, are surprisingly outstandingly suitable for preparing NMR diagnostic agents without showing the disadvantages mentioned.

The cascade polymer complexes according to the invention can be described by general formula I

R-L-A-{X—[Y-(Z-{W—K_{w}}_{z})_{y}]ₓ}ₐ₋₁ (I)
where
R=is an HSA-binding unit,
L=is a linker or a bond,
A=is a nitrogen-containing cascade core of base multiplicity a,
X and Y=are independently of one another a direct linkage or a cascade reproduction unit of reproduction multiplicity x and y, respectively,
Z and W=are independently of one another a direct linkage or a cascade reproduction unit of reproduction multiplicity z and w, respectively,
K is the residue of a complexing agent,
a=is numbers 2 to 12, and
x, y, z and w=are independently of one another the numbers 1 to 4,
with the proviso that exactly one multiplicity of the base multiplicity a of the cascade core A represents exactly one point of linkage to L, and
with the proviso that the cascade polymer complexes comprise in the complexing agent residue K in total at least 4 ions of an element of atomic number 20 to 29, 39, 42 to 44 or 57 to 83 and comprise where appropriate cations of inorganic and/or organic bases, amino acids or amino amides.

Preferred compounds according to formula I are those characterized in that the following applies to the product of the multiplicities

4≦(a−1)*x*y*z*w≦64.

Particularly preferred compounds according to formula I are those characterized in that the following applies to the product of the multiplicities

8≦(a−1)*x*y*z*w≦48.

The following are suitable as cascade core A:

Nitrogen atom,

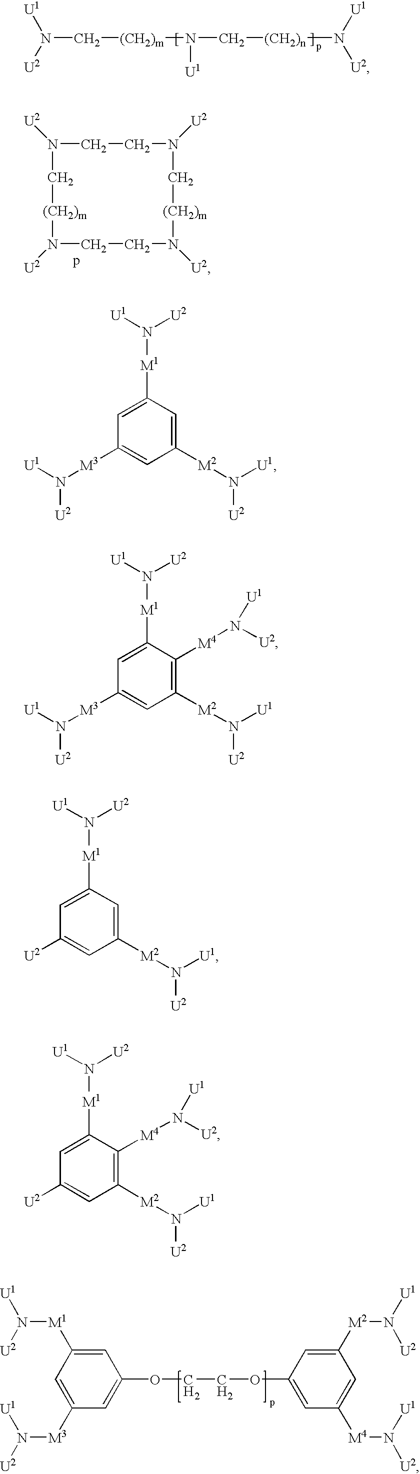

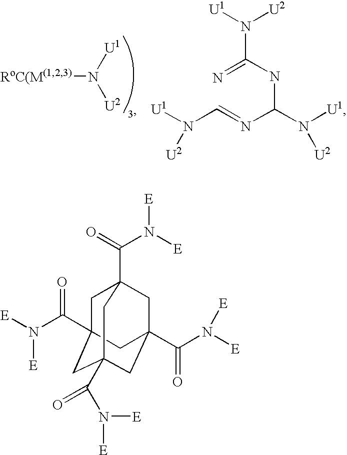

in which
m and n are the numbers 1 to 10,
p is the numbers 0 to 10,
U¹ is Q¹ or E,
U² is Q² or E with
   E meaning the group

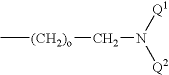

where
o is the numbers 1 to 6,
Q¹ is a hydrogen atom or Q² and
Q² is a direct linkage
M¹, M², M³, M⁴ are independently of one another a direct linkage, a C₁-C₁₀-alkylene chain which is optionally interrupted by 1 to 3 oxygen atoms and/or is optionally substituted by 1 to 2 oxo groups,
M^{(1,2,3)} is meant to denote that in each of the three occurrences of M, M can be selected independently from each other from the common definition for M¹, M², . . . (in other words according to this formula, M can have three times the same meaning or can be different for two or each of the three occurrences),
R^{o} is a branched or unbranched C₁-C₁₀-alkyl radical, a nitro, amino, carboxylic acid group or is

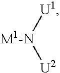

where the number of Q² corresponds to the base multiplicity a, and with the proviso that exactly one Q² represents a linkage to L.

The simplest case of a cascade core is represented by the nitrogen atom whose three bonds (base multiplicity a=3) are occupied in a first “inner layer” (generation 1) by two reproduction units X and Y (when X is a direct linkage) or Z (when X and Y each represent a direct linkage); in other words: the three hydrogen atoms of the underlying cascade starter ammonia A(H)ₐ═NH₃ have been replaced by two reproduction units X and Y or Z and by a direct linkage to L. The number of Q² (where Q² occurs in the respective cascade core A) present in the examples described above for the cascade core A represents the base multiplicity a.

The reproduction units X, Y, Z and W comprise —NQ¹Q² groups in which Q¹ is a hydrogen atom or Q² and Q² is a direct linkage. The number of Q² present in the respective reproduction unit (e.g. X) corresponds to the reproduction multiplicity of this unit (e.g. x in the case of X). The product of the multiplicities (a−1 )·x·y·z·w indicates the number of complexing agent residues K linked in the cascade polymer. The polymers according to the invention comprise at least 4 and at most 64 K residues in the molecule, which are in each case able to bind one to a maximum of three (in the case of divalent ions), preferably one ion, of an element of the abovementioned atomic numbers.

The last generation, i.e. the reproduction unit W linked to the complexing agent residues K is linked via NH groups (—NQ¹Q² with Q¹ meaning a hydrogen atom and Q²=direct linkage) to K, whereas the preceding reproduction units may be connected together both via NHQ² groups (e.g. by acylation reactions) and via NQ²Q² groups (e.g. by alkylation reactions).

The cascade polymer complexes according to the invention have a maximum of 10 generations (i.e. it is also possible for more than in each case only one of the reproduction units X, Y and Z to be present in the molecule), but preferably 2 to 5 generations, with at least two of the reproduction units in the molecule being different.

Cascade cores A which may be mentioned as preferred are those covered by the abovementioned general formulae when
m is the numbers 1-3, particularly preferably the number 1,
n is the numbers 1-3, particularly preferably the number 1,
p is the numbers 0-3, particularly preferably the number 1,
o is the number 1,
M¹, M², M³, M⁴ is independently one another a direct linkage, —CH₂—, —CO— or —CH₂CO— group and
R^{o} is a —CH₂NU¹U²-, CH₃— or NO₂ group.

Further preferred cascade starters A which may be mentioned are for example:

Tris(aminoethyl)amine,

Tris(aminopropyl)amine,

Diethylenetriamine,

Triethylenetetramine,

Tetraethylenepentamine,

1,3,5-Tris(aminomethyl)benzene,

Trimesamide,

Aminoisophthalamide,

3,5-Bis(2-aminoethoxy)benzamide,

3,5-Bis(3-aminopropoxy)benzamide,

3,5-Bis(2-aminoethoxy)aniline,

3,5-Bis(3-aminopropoxy)aniline,

3,4,5-Tris(2-aminoethoxy)benzamide,

3,4,5-Tris(3-aminopropoxy)benzamide,

3,4,5-Tris(2-aminoethoxy)aniline,

3,4,5-Tris(3-aminopropoxy)aniline,

3,5-Diamino-1-benzamide,

1,4,7-Triazacyclononane,

1,4,7,10-Tetraazacyclododecane,

1,4,7,10,13-Pentaazacyclopentadecane,

1,4,8,11-Tetraazacyclotetradecane,

1,4,7,10,13,16-Hexaazacyclooctadecane,

1,4,7,10,13,16,19,22,25,28-Decaazacyclotriacontane,

Tetrakis(aminomethyl)methane,

1,1,1-Tris(aminomethyl)ethane,

Tris(aminopropyl)nitromethane,

2,4,6-Triamino-1,3,5-triazine,

Lysinamide,

Ornithinamide,

Glutamamide,

Aspartamide,

Diaminopropanoamide.

A in particularly preferred compounds according to formula I is selected from:

Aminoisophthalamide,

3,5-Bis(2-aminoethoxy)benzamide,

3,5-Bis(3-aminopropoxy)benzamide,

3,5-Bis(2-aminoethoxy)aniline,

3,5-Bis(3-aminopropoxy)aniline,

3,4,5-Tris(2-aminoethoxy)benzamide,

3,4,5-Tris(3-aminopropoxy)benzamide,

3,4,5-Tris(2-aminoethoxy)aniline,

3,4,5-Tris(3-aminopropoxy)aniline,

3,5-Diamino-1-benzamide,

Lysinamide,

Ornithinamide,

Glutamamide,

Aspartamide,

Diaminopropanoamide.

It may be pointed out that the definition as cascade core A and thus the separation of cascade core and first reproduction unit or linker can be chosen purely formally and thus irrespective of the actual synthetic structure of the desired cascade polymer complexes. Thus, for example, tris(aminoethyl)amine can be regarded both itself as cascade core A (compare the first general formula indicated for A with m=n=p=1, U¹=E with o meaning the number 1 and U¹=U²=Q²) but also as nitrogen atom (=cascade core A) which has as first generation two reproduction units and a connection to the linker with the following structural formula in each case

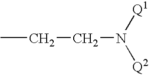

(compare the definition of E).

The cascade reproduction units X, Y, Z and W are determined independently of one another by
E,

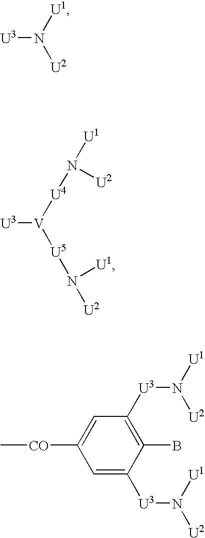

in which,
U¹ is Q¹ or E,
U² is Q² or E with
E meaning the group

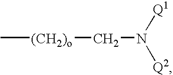

where
o is the numbers 1 to 6,
Q¹ is a hydrogen atom or Q²,
Q² is a direct linkage,
U³ is a C₁-C₂₀-alkylene chain which is optionally interrupted by 1 to 10 oxygen atoms and/or 1 to 2-N(CO)_{q}—R²—, 1 to 2 phenylene and/or 1 to 2 phenyleneoxy radicals, and/or is optionally substituted by 1 to 2 oxo, thioxo, carboxy, C₁-C₅-alkyl-carboxy, C₁-C₅-alkoxy, hydroxy, C₁-C₅-alkyl groups,
   where
   q is the numbers 0 or 1, and
   R² is a hydrogen atom, a methyl or an ethyl radical, which is optionally substituted by 1-2 hydroxy or 1 carboxy group(s),
   B is a hydrogen atom or the group

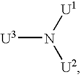

V is the methine group

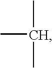

when at the same time U⁴ is a direct linkage or the group M, and U⁵ has one of the meanings of U³, or
V is one of the following groups

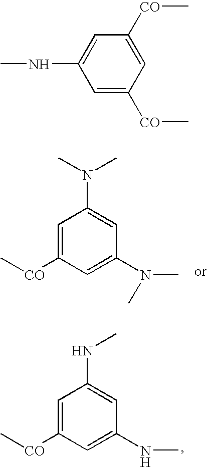

when at the same time U⁴ and U⁵ are identical and are the direct linkage or the group M,
where M is a direct linkage or a C₁-C₁₀-alkylene chain which is optionally interrupted by 1 to 3 oxygen atoms and/or is optionally substituted by 1 to 2 oxo groups.

Preferred cascade reproduction units X, Y, Z and W are those in which in the abovementioned general formulae

the radical U³ is a direct linkage, —CO—, —COCH₂OCH₂CO—, —COCH₂—, —CH₂CH₂—, —CONHC₆H₄—, —COCH₂CH₂CO—, —COCH₂—CH₂CH₂CO—, or —COCH₂CH₂CH₂CH₂CO—,

the radical U⁴ is a direct linkage or is —CH₂CO—,

the radical U⁵ is a direct linkage, is —(CH₂)₄—, —CH₂CO—, —CH(COOH)—, —CH₂OCH₂CH₂—, —CH₂C₆H₄—, CH₂—C₆H₄OCH₂CH₂—,

the radical E is a group

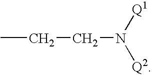

Examples which may be mentioned of the said cascade reproduction units X, Y, Z and W are:

—CH₂CH₂NH—; —CH₂CH₂N<; —CO—(CH₂)₂—NH—; —CO—(CH₂)₃—NH—; —CO—(CH₂)₄—NH—; —CO—(CH₂)₅—NH—; —CO—(CH₂)₆—NH—;

—CO—(CH₂)₂—N<; —CO—(CH₂)₃—N<; —CO—(CH₂)₄—N<; —CO—(CH₂)₅—N<; —CO—(CH₂)₆—N<;

—COCH(NH—)(CH₂)₄NH—; —COCH(N<)(CH₂)₄N<;

—COCH₂OCH₂CON(CH₂CH₂NH—)₂; —COCH₂OCH₂CON(CH₂CH₂N<)₂;

—COCH₂N(CH₂CH₂NH—)₂; —COCH₂N(CH₂CH₂N<)₂;

—COCH₂NH—; —COCH₂N<;

—COCH₂CH₂CON(CH₂CH₂NH—)₂; —COCH₂CH₂CON(CH₂CH₂N<)₂;

—COCH₂OCH₂CONH—C₆H₄—CH[CH₂CON(CH₂CH₂NH—)₂]₂;

—COCH₂OCH₂CONH—C₆H₄—CH[CH₂CON(CH₂CH₂N<)₂]₂;

—COCH₂CH₂CO—NH—C₆H₄—CH[CH₂CON(CH₂CH₂NH—)₂]₂;

—COCH₂CH₂CO—NH—C₆H₄—CH[CH₂CON(CH₂CH₂N<)₂]₂;

—CONH—C₆H₄—CH[CH₂CON(CH₂CH₂NH—)₂]₂;

—CONH—C₆H₄—CH[CH₂CON(CH₂CH₂N<)₂]₂;

—COCH(NH—)CH(COOH)NH—; —COCH(N<)CH(COOH)N<;

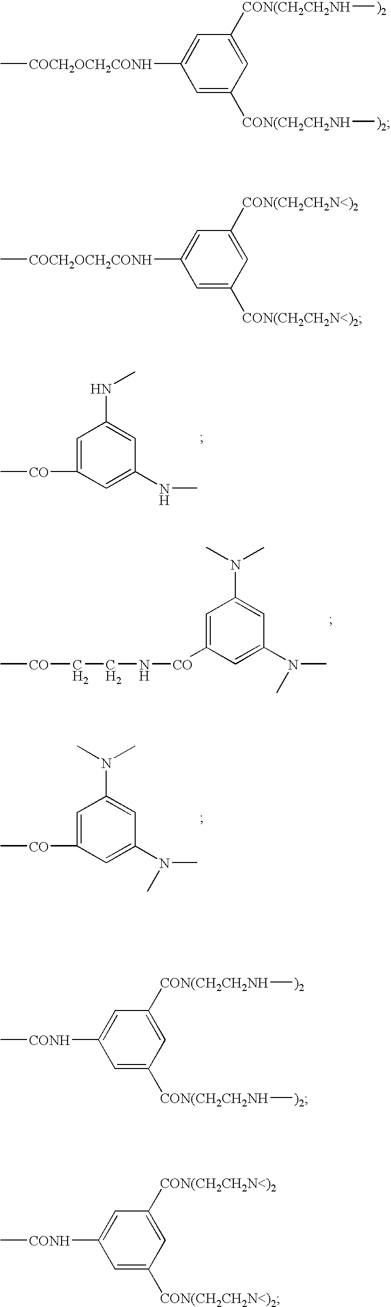

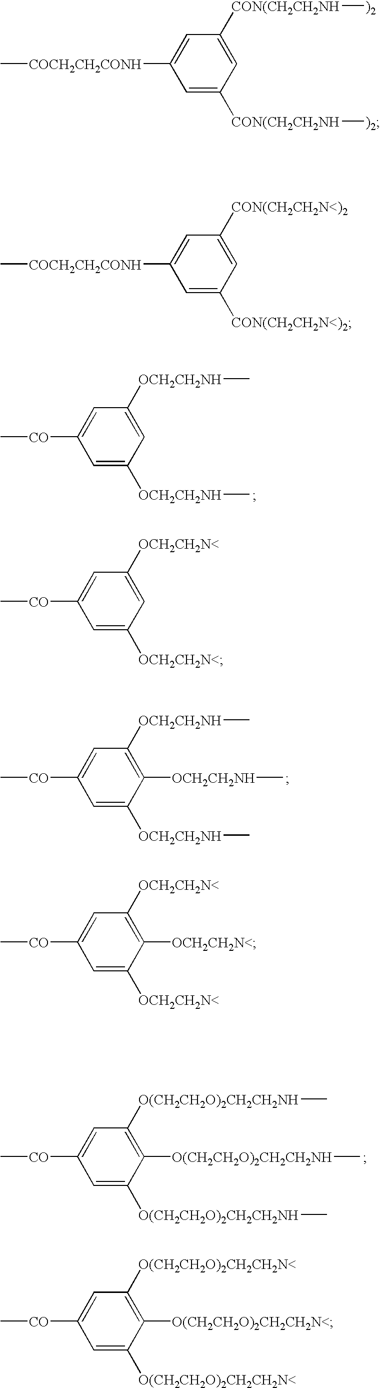

The complexing agent residues K are described by the general formulae IA, IB and IC:

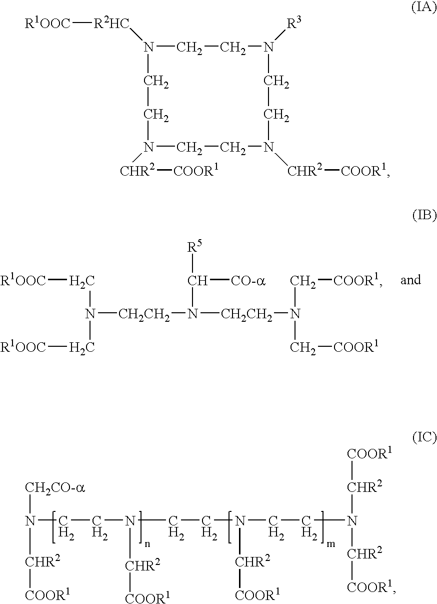

in which
n and m are each the numbers 0, 1, 2, 3 or 4, and where the total of n plus m is not larger than 4,
R¹ are independently of one another a hydrogen atom or a metal ion equivalent of atomic numbers 20-29, 39, 42-44 or 57-83,
R² is a hydrogen atom, a methyl or an ethyl radical, which is optionally substituted by 1-2 hydroxy or 1 carboxy group(s),
R³ is a

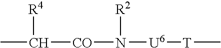

group or a

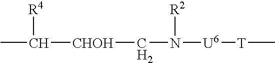

group,
R⁴ is isopropyl, cyclohexyl, a straight-chain, branched, saturated or unsaturated C₁-C₃₀-alkyl chain which is optionally interrupted by 1-10 oxygen atoms, 1 phenylene, 1 phenyleneoxy groups and/or is optionally substituted by 1-5 hydroxy, 1-3 carboxy, 1 phenyl group(s),
R⁵ is a hydrogen atom or is R⁴,
U⁶ is a straight-chain, branched, saturated or unsaturated C₁-C₂₀-alkylene group which optionally comprises 1-5 imino, 1-3 phenylene, 1-3 phenyleneoxy, 1-3 phenyleneimino, 1-5 amide, 1-2 hydrazide, 1-5 carbonyl, 1-5 ethyleneoxy, 1 urea, 1 thiourea, 1-2 carboxyalkylimino, 1-2 ester groups, 1-10 oxygen, 1-5 sulphur and/or 1-5 nitrogen atom(s) and/or is optionally substituted by 1-5 hydroxy, 1-2 mercapto, 1-5 oxo, 1-5 thioxo, 1-3 carboxy, 1-5 carboxyalkyl, 1-5 ester and/or 1-3 amino group(s), where the phenylene groups which are optionally present may be substituted by 1-2 carboxy, 1-2 sulphone or 1-2 hydroxy groups,
T is a —CO-α, —NHCO-α or —NHCS-α group and
α is the point of linkage to the terminal nitrogen atoms of the last generation of the reproduction unit W.

Complexing agent residues K which may be mentioned as preferred are those in which the C₁-C₂₀, preferably C₁-C₁₂, alkylene chain representing U⁶ in the formula IA indicated above comprises the groups —CH₂—, —CH₂NHCO—, —NHCOCH₂O—, —NHCOCH₂OC₆H₄—, —N(CH₂CO₂H)—, —NHCOCH₂C₆H₄—, —NHCSNHC₆H₄—, —CH₂OC₆H₄—, —CH₂CH₂O—, and/or is substituted by the groups —COOH, —CH₂COOH.

Examples which may be mentioned of U⁶ are the following groups: —CH₂—, —CH₂CH₂—, —CH₂CH₂CH₂—, —C₆H₄—, —C₆H₁₀—, —CH₂C₆H₅—,

—CH₂NHCOCH₂CH(CH₂CO₂H)—C₆H₄—, —CH₂NHCOCH₂OCH₂—, —CH₂NHCOCH₂C₆H₄—,

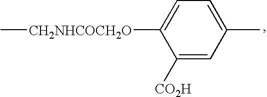

—CH₂NHCSNH—C₆H₄—CH(CH₂COOH)CH₂—, —CH₂OC₆H₄—N(CH₂COOH)CH₂—,

—CH₂NHCOCH₂O(CH₂CH₂O)₄—C₆H₄—, —CH₂O—C₆H₄—,

—CH₂CH₂—O—CH₂CH₂—, —CH₂CH₂—O—CH₂CH₂—O—CH₂CH₂—,

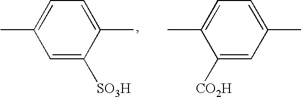

Examples of R⁴ which may be indicated are the following groups:

isopropyl, cyclohexyl, —CH₃, —C₆H₅, —CH₂—COOH, —CH₂—C₆H₅, —CH₂—O—(CH₂CH₂—O—)₆CH₃, —CH₂—OH.

If the agent according to the invention is intended for use in NMR diagnosis, the central ion of the complex salt must be paramagnetic. These are in particular the divalent and trivalent ions of the elements of atomic numbers 21-29, 42, 44 and 58-70. Examples of suitable ions are the chromium(III), iron(II), cobalt(II), nickel(II), copper(I), praseodymium(III), neodymium(III), samarium(III) and ytterbium(III) ions.

Because of their very strong magnetic moment, the gadolinium(III), terbium(III), dysprosium(III), holmium(III), erbium(III), manganese(II) and iron(III) ions are particularly preferred.

The function of the structure L is to connect together the two functional units R and A. L may in this connection be a direct linkage or a linker. The term linker for the purposes of this invention includes any chemical structure which is linked on one side covalently to the HSA-binding unit R and on the other side to the nitrogen-containing cascade core A, and thus connects R to A. The meaning of the term linker is thus functionally defined and includes a large number of widely different chemical compounds. The relevant skilled person is able on the basis of his expert knowledge without undue burden to synthesize a large number of very different linker structures which comply with the function according to the invention of connecting R to A. The skilled person merely needs to carry out routine experiments for this purpose.

Preferred linker structures L include a direct linkage, straight-chain or branched, saturated or unsaturated carbon chains having 1 to 30 carbon atoms which may be interrupted and/or substituted. If the carbon chains of the linker are interrupted, they are preferably interrupted by one or more cyclic or heterocyclic carbon groups having 3 to 8 carbon atoms or by one or more oxygen, nitrogen, sulphur and/or phosphorus atoms which themselves optionally may have further atoms such as, for example, hydrogen or oxygen, or groups, linked. Linkers in the sense of this invention may also include one or more amino acids.

In particularly preferred compounds according to formula I, L is selected from:

a direct linkage,

—O—CH₂—CO—NH—(CH₂₋CH₂₋O)₁₋₁₀—CH₂—CH₂—CO—,

—O—CH₂—CO—,

—O—CH₂—CO—NH—_{C1-12}—CO—,

—CO—,

—OP(O₂)O—C₁₋₁₂—CO—,

—O—CH₂—CO-Pro₄-,

—O—CH₂—CO—NH-aryl-C≡C-aryl-CO—,

—O—CH₂—CO—NH-aryl-C≡C—C≡C-aryl-CO—,

—CO—NH—CH₂—CH₂—,

where Pro is the amino acid proline.

The linkers L are oriented in this case as indicated below:

R—O—CH₂—CO—NH—(CH₂₋CH₂₋O)₁₋₁₀—CH₂—CH₂—CO-A,

R—O—CH₂—CO-A,

R—O—CH₂—CO—NH—C₁₋₁₂—CO-A,

R—CO-A,

R—OP(O₂)O—C₁₋₁₂—CO-A,

R—O—CH₂—CO-Pro₄-A,

R—CO—NH—CH₂—CH₂-A,

R—O—CH₂—CO—NH-aryl-C≡C-aryl-CO-A.

The compounds according to the invention according to formula I include an HSA binding unit R which is a chemical structure which binds to the protein human serum albumin (HSA) and has a direct linkage to L.

In preferred compounds according to formula I, R has a molecular weight which is not greater than 2000 Da.

In particularly preferred compounds according to formula I, R has at least one particular binding affinity to HSA, the inhibition constant Ki being less than or equal to 50 μM, measured by the method which is described in Example 3 and is from the US patent application with the publication number US 2004/0254119 (West et al., U.S. application Ser. No. 10/487,025).

It is particularly preferred for R to have a Ki of less than or equal to 15 μM.

Examples which may be mentioned of suitable HSA-binding groups are R:

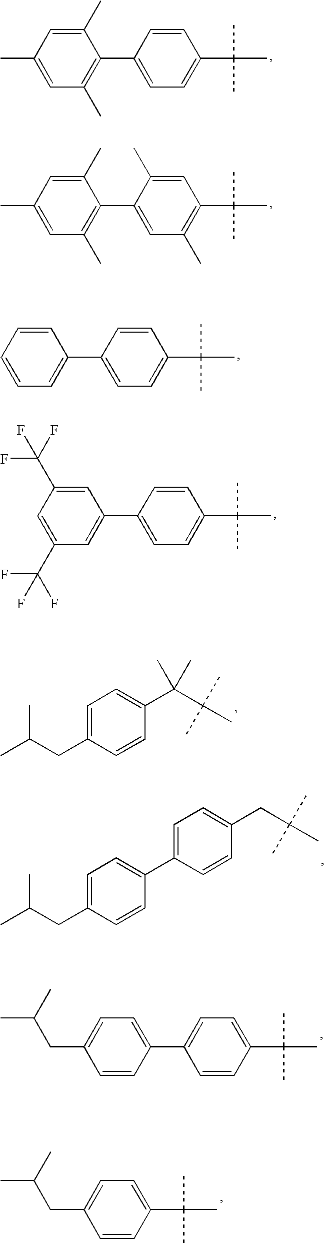

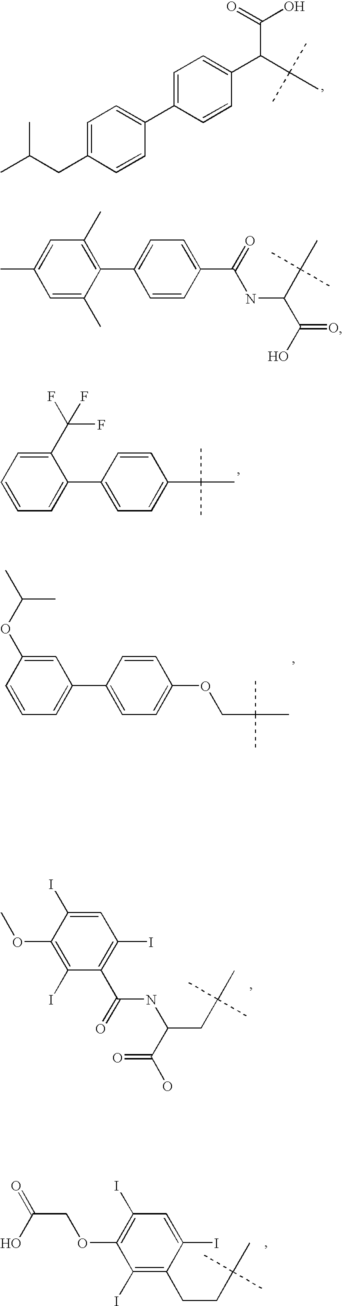

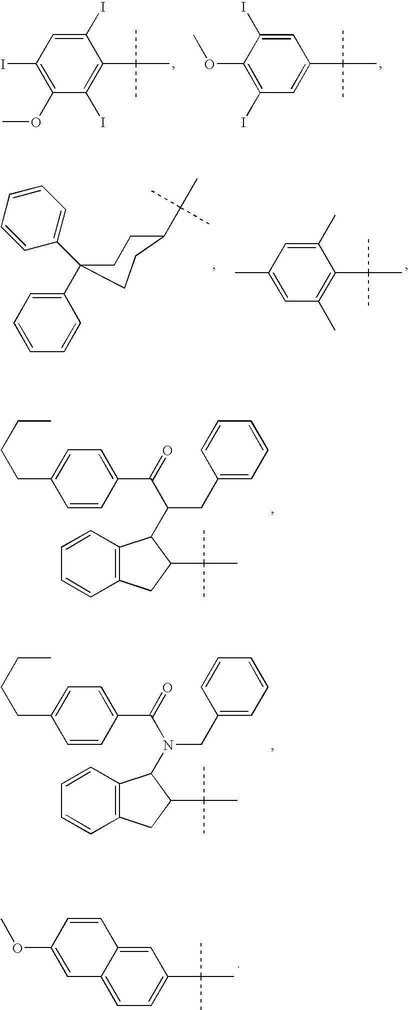

The cascade polymer complexes according to the invention comprise at least 4 ions of an element of the abovementioned atomic numcalculated

Preferred compounds of the formula I comprise at least 8 ions of an element of the abovementioned atomic numcalculated

The remaining acidic hydrogen atoms, meaning those which have not been replaced by the central ion, may optionally be replaced in whole or in part by cations of inorganic and/or organic bases, amino acids or amino amides.

Suitable inorganic cations are, for example, the lithium ion, the potassium ion, the calcium ion, the magnesium ion and in particular the sodium ion. Suitable cations of organic bases are inter alia those of primary, secondary or tertiary amines such as, for example, ethanolamine, diethanolamine, morpholine, glucamine, N,N-dimethylglucamine and in particular N-methyl-glucamine. Suitable cations of amino acids are, for example, those of lysine, of arginine and of omithine, and the amides of otherwise acidic or neutral amino acids.

The compounds according to the invention are distinguished by a high blood concentration in particular at certain times. This is advantageous in the choice of suitable imaging times and permits the signal-to-background ratio to be more favourable, especially at early and intermediate imaging times, compared with compounds like those described in European patent EP 0 836 485.

The compounds according to the invention are particularly suitable for use as coronary angiography contrast media and in NMR applications using higher magnetic field strengths such as, for example 1.5 or 3 Tesla.

The compounds according to the invention, which have a molecular weight of 5000-60 000 Da, preferably 5000-40 000 Da, have the desired properties described. They comprise, stably bound in the complex, the large number of metal ions required for their use.

They accumulate in areas of increased vascular permeability such as, for example, in tumours, permit statements to be made about the perfusion of tissues, provide the possibility of determining the blood volume in tissues, of selectively shortening the relaxation times or densities of the blood, and of visualizing the permeability of the blood vessels in images. Such physiological information cannot be obtained through the use of extracellular contrast media such as, for example, Gd-DTPA [Magnevist®]. From these aspects also emerge the areas of use in the modern imaging methods of magnetic resonant imaging and computed tomography: more specific diagnosis of malignant tumours, early check of therapy in cases of cytostatic, antiinflammatory or vasodilatory therapy, early identification of areas of reduced perfusion (e.g. in the myocardium), angiography for vascular disorders, and identification and diagnosis of (sterile or infectious) inflammations.

The cascade polymer complexes according to the invention have surprising properties by comparison with known cascade polymer complexes like those described in European patent EP 0 836 485. These surprising properties permit an even more flexible choice of the imaging times and a more favourable signal-to-background ratio, particularly at certain imaging times. It is particularly surprising by comparison with the known cascade polymer complexes from EP 0 836 485 especially that, although the cascade polymer complexes according to the invention presented here have one polymer arm less than the known cascade complexes, and thus tend to be smaller by comparison, and thus ought to be more prone to extravasation, the novel cascade polymer complexes according to the invention in fact show a distinctly improved residence time in the blood.

The cascade polymer complexes according to the invention are also outstandingly suitable for (interstitial and i.v.) lymphography.

Further advantages which must be emphasized by comparison with known contrast media such as, for example, Gd-DTPA [Magnevist®] is the greater efficiency as contrast media for magnetic resonance imaging (higher relaxivity), leading to a distinct reduction in the dose necessary for diagnosis. At the same time, the contrast media according to the invention can be formulated as solutions which are isoosmolar with blood and thus reduce the osmotic stress on the body, as reflected by a reduced toxicity of the substance (higher toxic threshold). Lower doses and higher toxic threshold lead to a significant increase in the safety of contrast media applications in modern imaging methods.

By comparison with macromolecular contrast media based on carbohydrates, e.g. dextran (European patent application, publication No. 0 326 226), which—as mentioned—usually carry only about 5% of the signal-enhancing paramagnetic cation, the polymer complexes according to the invention usually have a content of about 20% of the paramagnetic cation. The macromolecules according to the invention thus bring about a very much greater signal enhancement per molecule, simultaneously leading to the dose necessary for magnetic resonance imaging being considerably smaller than for the macromolecular contrast media based on carbohydrates.

Compared with the other prior art polymeric compounds mentioned, the cascade polymer complexes according to the invention are distinguished by improved excretion behaviour, higher activity, greater stability and/or better tolerability.

A further advantage of the present invention is that complexes with hydrophilic or lipophilic, macrocyclic or open-chain, low molecular weight or high molecular weight ligands have now become available. It is thus possible to control the tolerability and pharmacokinetics of these polymer complexes by chemical substitution.

The cascade polymer complexes according to the invention are prepared by reacting compounds of the general formula I′

R-L-A-{X—[Y-(Z-{W-β_{w}{_{z})_{y}]ₓ{ₐ₋₁ (I′)
where
R=is an HSA-binding unit,
L=is a linker or a bond,
A=is a nitrogen-containing cascade core of base multiplicity a,
X and Y=are independently of one another a direct linkage or a cascade reproduction unit of reproduction multiplicity x and y, respectively,
Z and W=are independently of one another a direct linkage or a cascade reproduction unit of reproduction multiplicity z and w, respectively,
β=is the point of linkage of the terminal NH groups of the last generation of the reproduction unit W,
a is numbers 2 to 12, and
x, y, z and w are independently of one another the numbers 1 to 4,
with the proviso that exactly one base multiplicity a of the cascade core A represents exactly one point of linkage to L,
   with a complex or complexing agent K′ of the general formula I′A or I′B

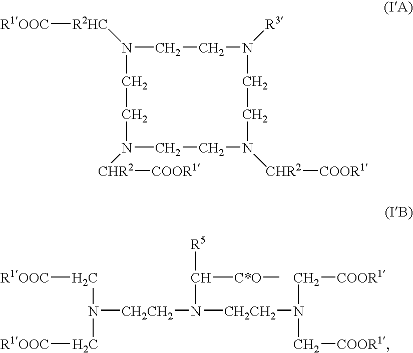

where
R^{1′} are independently of one another a hydrogen atom or a metal ion equivalent of atomic numbers 20-29, 39, 42-44 or 57-83, or an acid protective group,
R² is a hydrogen atom, a methyl or an ethyl radical, which is optionally substituted by 1-2 hydroxy or 1 carboxy group(s),
R^{3′} is a

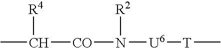

group or a

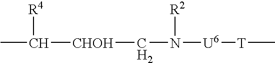

group,
R⁴ is isopropyl, cyclohexyl, a straight-chain, branched, saturated or unsaturated C₁-C₃₀-alkyl chain which is optionally interrupted by 1-10 oxygen atoms, 1 phenylene, 1 phenyleneoxy groups and/or is optionally substituted by 1-5 hydroxy, 1-3 carboxy, 1 phenyl group(s),
R⁵ is a hydrogen atom or is R⁴,
U⁶ is a straight-chain, branched, saturated or unsaturated C₁-C₂₀-alkylene group which optionally comprises 1-5 imino, 1-3 phenylene, 1-3 phenyleneoxy, 1-3 phenyleneimino, 1-5 amide, 1-2 hydrazide, 1-5 carbonyl, 1-5 ethyleneoxy, 1 urea, 1 thiourea, 1-2 carboxyalkylimino, 1-2 ester groups, 1-10 oxygen, 1-5 sulphur and/or 1-5 nitrogen atom(s) and/or is optionally substituted by 1-5 hydroxy, 1-2 mercapto, 1-5 oxo, 1-5 thioxo, 1-3 carboxy, 1-5 carboxyalkyl, 1-5 ester and/or 1-3 amino group(s), where the phenylene groups which are optionally present may be substituted by 1-2 carboxy, 1-2 sulphone or 1-2 hydroxy groups,
T′ is a —C*O—, —COOH—, —N═C═O— or —N═C═S— group and
C*O is an activated carbonyl group, and

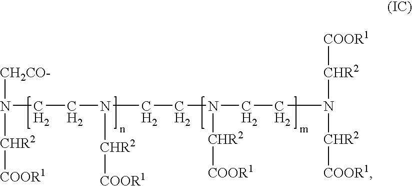

in which
n and m are each the numbers 0, 1, 2, 3 or 4, and where the total of n and m is not larger than 4,
R¹ and R² independently of one another may each have the abovementioned meaning, with the proviso that—if K′ is a complex—at least two (in the case of divalent metals) or three (in the case of trivalent metals) of the substituents R¹ are a metal ion equivalent of the abovementioned elements, and that if desired further carboxyl groups are present in the form of their salts with inorganic and/or organic bases, amino acids or amino amides, where appropriate eliminating protective groups which are present, reacting the cascade polymers obtained in this way—if K′ is a complexing agent—in a manner known per se with at least one metal oxide or metal salt of an element of atomic numbers 20-29, 39, 42, 44 or 57-83, and where appropriate subsequently replacing acidic hydrogen atoms which are still present in the cascade polymer complexes obtained in this way in whole or in part by cations of inorganic and/or organic bases, amino acids or amino amides, and where appropriate acylating free terminal amino groups which are still present if desired—before or after the metal complexation.

Preferred complexing agents K′ have the following general formula I′A:

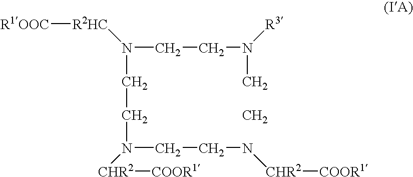

where
R^{1′} are independently of one another a hydrogen atom or a metal ion equivalent of atomic numbers 20-29, 39, 42-44 or 57-83,
R² is a hydrogen atom, a methyl or an ethyl radical, which is optionally substituted by 1-2 hydroxy or 1 carboxy group(s),
R^{3′} is a

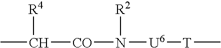

group or a

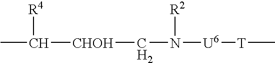

group
R⁴ is isopropyl, cyclohexyl, a straight-chain, branched, saturated or unsaturated C₁-C₃₀-alkyl chain which is optionally interrupted by 1-10 oxygen atoms, 1 phenylene, 1 phenyleneoxy groups and/or is optionally substituted by 1-5 hydroxy, 1-3 carboxy, 1 phenyl group(s), in particularly preferred embodiments R⁴ is selected from isopropyl and cyclohexyl,
U⁶ is a straight-chain, branched, saturated or unsaturated C₁-C₂₀-alkylene group which optionally comprises 1-5 imino, 1-3 phenylene, 1-3 phenyleneoxy, 1-3 phenyleneimino, 1-5 amide, 1-2 hydrazide, 1-5 carbonyl, 1-5 ethyleneoxy, 1 urea, 1 thiourea, 1-2 carboxyalkylimino, 1-2 ester groups, 1-10 oxygen, 1-5 sulphur and/or 1-5 nitrogen atom(s) and/or is optionally substituted by 1-5 hydroxy, 1-2 mercapto, 1-5 oxo, 1-5 thioxo, 1-3 carboxy, 1-5 carboxyalkyl, 1-5 ester and/or 1-3 amino group(s), where the phenylene groups which are optionally present may be substituted by 1-2 carboxy, 1-2 sulphone or 1-2 hydroxy groups,
T′ is a —C*O—, —COOH—, —N═C═O— or —N═C═S— group and
C*O is an activated carboxyl group.

They serve as important intermediates for preparing the cascade polymer complexes of the general formula I.

Mention may be made as example of an activated carbonyl group C*O in the complexes or complexing agents K′ of anhydride, p-nitrophenyl ester, N-hydroxysuccinimide ester, pentafluorophenyl ester and acid chloride.

The addition or acylation carried out to introduce the complexing agent units is carried out with substrates which comprise the desired substituent K (possibly linked to a leaving group), or from which the desired substituent can be generated by the reaction.

Examples of addition reactions which may be mentioned are reaction of isocyanates and isothiocyanates, carrying out the reaction of isocyanates preferably in aprotic solvents such as, for example, THF, dioxane, DMF, DMSO, methylene chloride at temperatures between 0 and 100° C., preferably between 0 and 50° C., where appropriate with the addition of an organic base such as triethylamine, pyridine, lutidine, N-ethyldiisopropylamine, N-methylmorpholine. The reaction with isothiocyanates is normally carried out in solvents such as, for example, water or lower alcohols such as, for example, methanol, ethanol, isopropanol or mixtures thereof, DMF or mixtures of DMF and water at temperatures between 0 and 100° C., preferably between 0 and 50° C., where appropriate with the addition of an organic or inorganic base such as, for example, triethylamine, pyridine, lutidine, N-ethyldiisopropylamine, N-methylmorpholine or alkaline earth metal, alkali metal hydroxides such as, for example, lithium, sodium, potassium, calcium hydroxide or their carbonates such as, for example, magnesium carbonate.

Examples of acylation reactions which may be mentioned are reaction of free carboxylic acids by methods known to the skilled person [e.g. J. P. Greenstein, M. Winitz, Chemistry of the Amino Acids, John Wiley & Sons, N.Y. (1961), pp. 943-945]. However, it has proved to be advantageous to convert the carboxylic acid group before the acylation reaction into an activated form such as, for example, anhydride, active ester or acid chloride [e.g. E. Gross, J. Meienhofer, The Peptides, Academic Press, N.Y. (1979), Vol. 1, pp. 65-314; N. F. Albertson, Org. React. 12, 157 (1962)].

In the case of reaction with active ester, reference may be made to the literature known to the skilled person [e.g. Houben-Weyl, Methoden der organischen Chemie, Georg Thieme Verlag, Stuttgart, Volume E 5 (1985), 633]. It can be carried out under the conditions indicated above for the anhydride reaction. However, it is also possible to use aprotic solvents such as, for example, methylene chloride, chloroform.

In the case of the acid chloride reactions, only aprotic solvents such as, for example, methylene chloride, chloroform, toluene or THF are used at temperatures between −20 to 50° C., preferably between 0 to 30° C. Reference may furthermore be made to the literature known to the skilled person [e.g. Houben-Weyl, Methoden der Organischen Chemie, Georg-Thieme-Verlag, Stuttgart, (1974), Volume 15/2, pp. 355-364].

If R^{1′} is an acid protective group, suitable groups are lower alkyl, aryl and aralkyl, for example the methyl, ethyl, propyl, butyl, phenyl, benzyl, diphenylmethyl, triphenylmethyl, bis(p-nitrophenyl)methyl groups, and trialkylsilyl groups.

The elimination of the protective groups which is desired where appropriate takes place by methods known to the skilled person, for example by hydrolysis, hydrogenolysis, alkaline hydrolysis of the esters with alkali in aqueous-alcoholic solution at temperatures from 0° C. to 50° C., or in the case of tert-butyl esters with the aid of trifluoroacetic acid.

Terminal amino groups which are where appropriate incompletely acylated with ligand or complex can, if desired, be converted into amides or monoamides. Mention may be made by way of example of reaction with acetic anhydride, succinic anhydride or diglycolic anhydride.

The desired metal ions are introduced in a manner like that disclosed for example in German patent Offenlegungsschrift DE 34 01 052 by dissolving or suspending the metal oxide or a metal salt (for example the nitrate, acetate, carbonate, chloride or sulphate) of the element of atomic numbers 20-29, 42, 44, 57-83 in water and/or a lower alcohol (such as methanol, ethanol or isopropanol) and reacting with the solution or suspension of the equivalent amount of the complex-forming ligand and subsequently, if desired, replacing acidic hydrogen atoms present in the acid groups by cations of inorganic and/or organic bases, amino acids or amino amides.

Introduction of the desired metal ions can take place both at the stage of the complexing agent I′A or I′B, i.e. before the coupling to the cascade polymers, and after coupling of the unmetallated ligands I′A or I′B.

Neutralization takes place in this case with the aid of inorganic bases (for example hydroxides, carbonates or bicarbonates) of for example sodium, potassium, lithium, magnesium or calcium and/or organic bases such as inter alia primary, secondary and tertiary amines such as, for example, ethanolamine, morpholine, glucamine, N-methyl- and N,N-dimethylglucamine, and basic amino acids such as, for example, lysine, arginine and ornithine or of amides of originally neutral or acidic amino acids such as, for example, hippuric acid, glycineacetamide.

The neutral complex compounds can be prepared for example by adding to the acidic complex salts in aqueous solution or suspension sufficient of the desired bases to reach the neutral point. The resulting solution can then be evaporated to dryness in vacuo. It is frequently advantageous to precipitate the neutral salts which have formed by adding water-miscible solvents such as, for example, lower alcohols (methanol, ethanol, isopropanol and others), lower ketones (acetone and others), polar ethers (tetrahydrofuran, dioxane, 1,2-dimethoxyethane and others) and thus obtain crystals which are easy to isolate and straightforward to purify. It has proved to be particularly advantageous to add the desired base to the reaction mixture even during the complex formation and thus save one process step.

If the acidic complex compounds comprise a plurality of free acidic groups, it is often expedient to prepare neutral mixed salts which comprise both inorganic and organic cations as counter ions.

This can take place for example by reacting the complex-forming ligands in aqueous suspension or solution with the oxide or salt of the element providing the central ion and with half the amount of an organic base which is required for neutralization, isolating the complex salt which is formed, purifying it if desired, and then adding the required amount of inorganic base for complete neutralization. The sequence of base addition may also be reversed.

Purification of the cascade polymer complexes obtained in this way takes place, where appropriate after the pH has been adjusted by addition of an acid or base to pH 6 to 8, preferably about 7, preferably by ultrafiltration with membranes of suitable pore size (e.g. Amicon® XM30, Amicon® YM10, Amicon® YM3, Amicon® YM1) or gel filtration on, for example, suitable Sephadex® gels.

In the case of neutral complex compounds it is frequently advantageous to pass the polymeric complexes over an anion exchanger, for example IRA 67 (OH⁻ form) and where appropriate additionally over a cation exchanger, for example IRC 50 (H⁺ form) to remove ionic components.

Preparation of the cascade polymers having the terminal amino groups necessary for coupling to the complexing agent K′ (or else the corresponding metal-containing complexes) generally starts from nitrogen-containing cascade starters A(H)ₐ which can be purchased or can be prepared by or in analogy to methods known from the literature. Introduction of the X, Y, Z and W generations takes place by methods known from the literature [e.g. J. March, Advanced Organic Chemistry, 3^{rd} ed.; John Wiley & Sons, (1985), 364-381] by acylation or alkylation reactions with protected amines having the desired structures and comprising functional groups able to link to the cascade core, such as, for example, carboxylic acids, isocyanates, isothiocyanates or activated carboxylic acids (such as, for example, anhydrides, active esters, acid chlorides) or halides (such as, for example, chlorides, bromides, iodides), aziridine, mesylates, toslyates or other leaving groups known to the skilled person.

However, it may be emphasized here once again that differentiation between cascade core A and reproduction units or the connection to the linker L is purely formal. It may be synthetically advantageous not to use the formal cascade starter A(H)ₐ but to introduce the nitrogen atoms belonging by definition to the cascade core only together with the first generation.

Amine protective groups which may be mentioned are the benzyloxycarbonyl, tertiary butoxycarbonyl, trifluoroacetyl, fluorenylmethoxycarbonyl, benzyl and formyl groups familiar to the skilled person [Th. W. Greene, P. G. M Wuts, Protective Groups in Organic Syntheses, 2nd ed, John Wiley and Sons (1991), pp. 309-385]. After elimination of these protective groups, which likewise takes place by methods known from the literature, it is possible to introduce the next desired generation into the molecule. Besides this assembling, consisting in each case of two reaction stages (alkylation or acylation and protective group elimination), of one generation it is also possible to introduce two, e.g. X—[Y]ₓ or more generations e.g. X—[Y-(Z)_{y}]ₓ, simultaneously with likewise only two reaction stages. These multigeneration units are assembled by alkylation or acylation of unprotected amines having the structures of the desired reproduction units (“reproduction amine”) with a second reproduction amine whose amine groups are in protected form.

The compounds of the general formula A(H)a required as cascade starters can be purchased or can be prepared by or in analogy to methods known from the literature [e.g. Houben-Weyl, Methoden der Org. Chemie, Georg-Thieme-Verlag, Stuttgart (1957), Vol. 11/1; M. Micheloni et al., Inorg. Chem. (1985), 24, 3702; T. J. Atkins et al., Org. Synth., Vol. 58; (1978), 86-98; The Chemistry of Heterocyclic Compounds: J. S. Bradshaw et al., Aza-Crown-Macrocycles, John Wiley & Sons, N.Y. (1993)].

The reproduction amines comprising the abovementioned functional groups required to assemble the generations are prepared by or in analogy to the methods described in the experimental section or by processes known from the literature.

Examples which may be mentioned are:

N^{α},N^{ε}-Dibenzyloxycarbonyllysine p-nitrophenyl ester;

HOOC—CH₂OCH₂CO—N(CH₂CH₂NH—CO—O—CH₂C₆H₅)₂;

HOOC—CH₂N(CH₂CH₂NH—CO—O—CH₂C₆H₅)₂;

HOOC—CH₂CH₂CO—N(CH₂CH₂NH—COCF₃)₂;

HOOC CH₂OCH₂CONH—C₆H₄—CH[CH₂CON(CH₂CH₂NH—CO—O—CH₂C₆H₅)₂]₂;

O═C═N—C₆H₄—CH[CH₂CON(CH₂CH₂NH—CO—O—CH₂C₆H₅)₂]₂

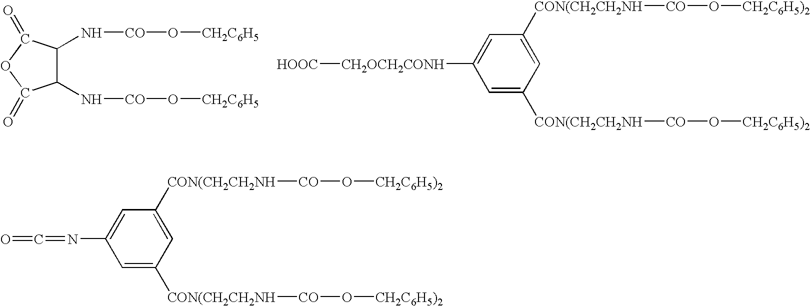

N-Benzyloxycarbonylaziridine can be prepared according to M. Zinic et al., J. Chem. Soc, Perkin Trans 1, 21-26 (1993)
N-Benzyloxycarbonylglycine can be purchased from, for example, Bachem Calif.

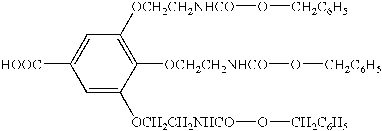

can be prepared according to C.J. Cavallito et al.,
   J. Amer. Chem. Soc. 1943, 65, 2140,
   by starting from N—CO—O—CH₂C₆H₅-(2-bromoethyl)amine
   [A. R. Jacobson et al., J. Med. Chem. (1991), 34, 2816]
   instead of benzyl chloride.

The complexes and complexing agents of the general formula I′A and I′B are prepared by or in analogy to the methods described in the experimental section or by methods known from the literature, see, for example, European patent applications No. 0 512 661, 0 430 863, 0 255 471 and 0 565 930.

Thus, compounds of the general formula I′A can be prepared for example by using as precursor of the functional group T′ a group T″, either in the meaning of a protected acid function which can be converted irrespective of the acid protective groups R¹ into the free-radical function by the processes detailed above, or in the meaning of a protected amine function which can be deblocked by processes known from the literature [Th. W. Greene, P. G. M. Wuts, Protective Groups in Organic Synthesis, 2nd edition, John Wiley & Sons (1991), pp. 309-385] and subsequently converted into the isocyanates or isothiocyanates [Methoden der Org. Chemie (Houben-Weyl), E 4, pp. 742-749, 837-843, Georg Thieme Verlag, Stuttgart, New York (1983)]. Such compounds can be prepared by or in analogy to the methods described in the experimental section by monoalkylation of cyclic compounds with suitable α-halo carboxamides [in aprotic solvents such as, for example, chloroform].

Compounds of the general formula I′B can be prepared for example by using as precursor of the activated carboxyl group —C*O— a protected acid function which can be converted irrespective of the acid protective groups R^{1′} into the free acid function by the processes detailed above and be activated by the processes known from the literature which are likewise described above. Such compounds can be prepared by or in analogy to the methods described in the experimental section or for example by reacting an amino acid derivative of the general formula II

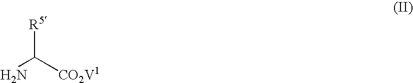

in which
R^{5′} has the meaning indicted for R⁵, with hydroxy or carboxyl groups present in R⁵ where appropriate being in protected form where appropriate, and
V¹ is a straight-chain or branched C₁-C₆-alkyl group, a benzyl, trimethylsilyl, triisopropylsilyl, 2,2,2,-trifluoroethoxy or 2,2,2-trichloroethoxy group, where V¹ is different from R^{1″}, with an alkylating agent of the general formula III

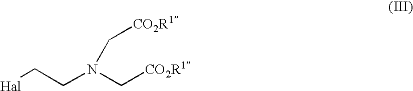

in which

R^{1″} is a protective group and

Hal is a halogen atom such as Cl, Br or I, but preferably Cl, [see also M. A. Williams, H. Rapoport, J. Org. Chem. 58, 1151 (1993)].

Preferred amino acid derivatives are the esters of naturally occurring α-amino acids.

Reaction of compound (II) with compound (III) preferably takes place in a buffered alkylation reaction, using an aqueous phosphate buffer solution as buffer. The reaction takes place at pH values of 7-9, but preferably at pH 8. The buffer concentration can be between 0.1-2.5 M, but a 2 M phosphate buffer solution is preferably used. The temperature of the alkylation can be between 0 and 50° C.; the preferred temperature is room temperature.

The reaction is carried out in a polar solvent such as, for example, acetonitrile, tetrahydrofuran, 1,4-dioxane or 1,2-dimethoxyethane. Acetonitrile is preferably used.

The pharmaceutical compositions according to the invention are likewise prepared in a manner known per se by suspending or dissolving the complex compounds according to the invention—where appropriate with admixture of additions customary in pharmaceutical technology—in aqueous medium and subsequently sterilizing the suspension or solution where appropriate. Suitable additions are, for example, physiologically acceptable buffers (such as, for example, trimethamine), additions of complexing agents or weak complexes (such as, for example, diethylenetriaminepentaacetic acid or the corresponding Ca cascade polymer complexes) or—if necessary—electrolytes such as, for example, sodium chloride or—if necessary—antioxidants such as, for example, ascorbic acid. If suspensions or solutions of the agents according to the invention in water or physiological saline are desired for enteral administration or other purposes, they are mixed with one or more excipient(s) customary in pharmaceutical technology [for example methylcellulose, lactose, mannitol] and/or surfactants [for example lecithins, Tween®, Myrj®] and/or flavouring(s) to adjust the taste [for example essential oils].

It is also possible in principle to prepare the pharmaceutical compositions according to the invention even without isolation of the complex salts. It is necessary to take particular care in every case to carry out the chelate formation in such a way that the salts and salt solutions according to the invention are virtually free of non-complexed metal ions with a toxic effect.

This can be ensured for example with the aid of coloured indicators such as xylenol orange by control titrations during the preparation process. The invention therefore also relates to processes for preparing the complex compounds and their salts. Purification of the isolated complex salt remains as final security.

The pharmaceutical compositions according to the invention preferably comprise 1 μmol-1.3 mol/l of the complex salt and are usually dosed in amounts of 0.0001-5 mmol/kg. They are intended for enteral and parenteral administration. The complex compounds according to the invention are used for NMR diagnosis in the form of their complexes with the ions selected from the elements with atomic numbers 21-29, 39, 42, 44 and 57-83.

The agents according to the invention comply with the diverse requirements for suitability as contrast media for magnetic resonance imaging. Thus, they are outstandingly suitable after oral or parenteral administration for improving the information provided by the image obtained with the aid of magnetic resonance imaging through increasing the signal intensity. They also show the high activity which is necessary in order to expose the body to minimum amounts of foreign substances, and the good tolerability which is necessary to maintain the non-invasive character of the investigations.

The agents according to the invention show a distinctly higher concentration in blood at relevant imaging times than the compounds described in the prior art, e.g. in European patent EP 0 836 485.

The good solubility in water and low osmolality of the agents according to the invention allows highly concentrated solutions to be prepared in order to keep the volume loading of the circulation within reasonable limits and to compensate for the dilution by a body fluid, which means that NMR diagnostic agents must be 100 to 1000 times more soluble in water than for NMR spectroscopy. In addition, the agents according to the invention show not only a high stability in vitro but also a surprisingly high stability in vivo, so that release or exchange of the—intrinsically toxic—ions which are not covalently bonded in the complexes takes place only extremely slowly during the time in which the novel contrast media are completely excreted again.

In general, the agents according to the invention are dosed for use as NMR diagnostic agents in amounts of 0.0001-5 mmol/kg, preferably 0.005-0.5 mmol/kg. Details of the use are discussed for example in H.-J. Weinmann et al., Am. J. of Roentgenology 142, 619 (1984).

Particularly low dosages (below 1 mg/kg of body weight) of organ-specific NMR diagnostic agents can be employed for example for detecting tumours and myocardial infarction.

The compounds according to the invention are distinguished by a high concentration in blood especially at particular times. This is advantageous in the choice of suitable imaging times and permits a more favourable signal-to-background ratio especially at early and intermediate imaging times compared with compounds like those described in European patent EP 0 836 485.

The compounds according to the invention are particularly suitable for use as coronary angiography contrast media and use in NMR diagnosis with higher magnetic fields such as, for example, 1.5 or 3 Tesla, as provided by modern NMR instruments.

The complex compounds according to the invention can furthermore be used advantageously as susceptibility reagents and as shift reagents for in vivo NMR spectroscopy.

The compounds according to the invention are surprisingly also suitable for differentiating malignant and benign tumours in regions without blood-brain barrier.

They are also distinguished by being completely eliminated from the body and are thus well tolerated.

Overall, it has been possible to synthesize novel complexing agents, metal complexes and metal complex salts which open up novel possibilities in diagnostic medicine.

The following examples serve to explain the subject-matter of the invention in more detail:

### EXAMPLE 1

### a) 2,5,2′,4′,6′-Pentamethylbiphenyl-4-ol

23.0 g (92.7 mmol) of 4-iodo-2,5-dimethylphenol (Alfa Chemicals Ltd.) are dissolved in 175 ml of tetrahydrofuran (THF) with exclusion of moisture. Then 5.36 g (4.64 mmol) of tetrakis-(triphenylphosphine)palladium(0) are added, and the mixture is heated to 65° C. At this temperature, 186 ml (186 mmol) of 1 M mesitylenemagnesium bromide in THF are added dropwise over the course of 30 min, and the mixture is stirred at this temperature for 1 h and at room temperature (RT) overnight.

The suspension is filtered with suction and washed with THF, and the solution is evaporated to dryness. The residue is taken up between diethyl ether and 1 M HCl, the phases are separated, and the aqueous phase is extracted twice with diethyl ether. The organic phase is dried over sodium sulphate, filtered and concentrated. The resulting crude product is chromatographed on silica gel (hexane/ethyl acetate gradient 98/2-80/20). The fractions containing the product are combined and evaporated.

Yield: 19.3 g (86.7% of theory)

Elemental Analysis:

| | | |
|---|---|---|
| calculated: | C 84.96 | H 8.39 |
| found: | C 85.17 | H 8.21 |

### b) tert-Butyl (2,5,2′,4′,6′-pentamethylbiphenyl-4-yloxy)acetate

19.3 g (80.3 mmol) of the phenol described in Example 1a are dissolved in dimethylformamide (DMF), and 22.2 g (160.6 mmol) of finely ground potassium carbonate are added. Then, at RT, 17.21 g (88.23 mmol) of tert-butyl bromoacetate are added dropwise. After stirring at RT overnight, salts are filtered off and the solution is evaporated to dryness in vacuo. The crude product is dissolved in ethyl acetate, and the organic phase is washed three times with water, dried over magnesium sulphate, filtered and concentrated.

Yield: 25.4 g (89.2% of theory)

Elemental Analysis:

| | | |
|---|---|---|
| calculated: | C 77.93 | H 8.53 |
| found: | C 77.68 | H 8.74 |

### c) (2,5,2′,4′,6′-Pentamethylbiphenyl-4-yloxy)acetic acid

25 g (70.5 mmol) of the ester described in Example 1b above are dissolved in 500 ml of methanol, and a solution of 28.2 g (705 mmol) of NaOH pellets in 250 ml of water is added, and the mixture is heated under reflux for 5 h. It is stirred at RT overnight, then the methanol is evaporated and the aqueous residue is adjusted to pH 5 with hydrochloric acid and extracted with ethyl acetate. The organic phase is washed twice with water, dried over sodium sulphate, filtered and concentrated.

Yield: 6.2 g (quantitative)

Elemental Analysis:

| | | |
|---|---|---|
| calculated: | C 76.48 | H 7.43 |
| found: | C 76.14 | H 7.20 |

### d) t-Butyl 3-(2-{2-[2-(2-{2-[2-(2-{2-[2-(2,5,2′,4′,6′-pentamethylbiphenyl-4-yloxy)acetylamino]-ethoxy}ethoxy)ethoxy]ethoxy}ethoxy)ethoxy]ethoxy}ethoxy)propionate

1.00 g (2 mmol) of amino-dPEG8™-t-butyl ester (Quanta Biodesign, Ltd.) are dissolved in 40 ml of DMF, mixed with 0.72 g (2.4 mmol) of (2,5,2′,4′,6′-pentamethylbiphenyl-4-yloxy)acetic acid (Example 1c) and, after addition of 0.78 g (6 mmol) of N,N-ethyldiisopropylamine and 0.91 g (2.4 mmol) of 2-(1H-benzotriazol-1-yl)tetramethyluroniumhexafluorophosphate (HBTU), stirred at RT overnight and concentrated. The residue is dissolved in dichloromethane and chromatographed on silica gel (dichloromethane/methanol 18:2).

Yield: 1.5 g (96.4% of theory)

Elemental Analysis:

| | | | |
|---|---|---|---|
| calculated: | C 64.84 | H 8.68 | N 1.80 |
| found: | C 64.67 | H 8.73 | N 1.69 |

### e) 3-(2-{2-[2-(2-{2-[2-(2-{2-[2-(2,5,2′,4′,6′-Pentamethylbiphenyl-4-yloxy)acetylamino]ethoxy}-ethoxy)ethoxy]ethoxy}ethoxy)ethoxy]ethoxy}ethoxy)propionic acid

1.48 g (1.9 mmol) of the tert-butyl ester described in Example 1d are dissolved in 60 ml (4 mmol) of 66.67 mM hydrogen chloride in diethyl ether, stirred at RT overnight, concentrated and then distilled several times with diethyl ether. The residue is employed in the following reaction without further characterization.

Yield: 1.4 g (quantitative)

### f) Bis{2-[2,6-bis(2,6-bis(benzyloxycarbonyl)aminohexanoylamino)hexanoylamino]ethyl}amine

7.0 g (7.5 mmol) of the N_{α},N_{ε}-bis(N,N′-dibenzyloxycarbonyllysyl)lysine (“tri-lysine”) described in Example 1c of EP 0836485, 1.2 g (7.5 mmol) of 1-hydroxybenzotriazole and 2.4 g (7.5 mmol) of 2-(1H-benzotriazol-1-yl)-1,1,3,3-tetramethyluronium tetrafluoroborate (TBTU; Peboc Limited, UK) are dissolved in DMF and stirred for 15 minutes. This solution is then mixed with 5.16 ml (30 mmol) of N-ethyldiisopropylamine and with 386 mg (3.75 mmol) of diethylenetriamine and stirred at room temperature overnight. After completion of the reaction, the residue after evaporation in vacuo is chromatographed with ethyl acetate/ethanol (2:1) on silica gel.

Yield: 5.8 g (79.5%)

Elemental Analysis:

| | | | |
|---|---|---|---|
| calculated: | C 64.21 | H 6.89 | N 10.80 |
| found: | C 64.02 | H 7.00 | N 10.56 |

### g) Carboxamide from 3-(2-{2-[2-(2-{2-[2-(2-{2-[2-(2,5,2′,4′,6′-pentamethylbiphenyl-4-yloxy)-acetylamino]ethoxy}ethoxy)ethoxy]ethoxy}ethoxy)ethoxy]ethoxy}ethoxy)propionic acid and bis{2-[2,6-bis(2,6-bis(benzyloxycarbonyl)aminohexanoylamino)hexanoylamino]ethyl}amine

681 mg (0.35 mmol) of bis{2-[2,6-bis(2,6-bis(benzyloxycarbonyl)aminohexanoylamino)-hexanoylamino]ethyl}amine (Example 1f) are dissolved in 10 ml of DMF, and 253 mg (0.35 mmol) of the 3-(2-{2-[2-(2-{2-[2-(2-{2-[2-(2,5,2′,4′,6′-pentamethylbiphenyl-4-yloxy)-acetylamino]ethoxy}ethoxy)ethoxy]ethoxy}ethoxy)ethoxy]ethoxy}ethoxy)propionic acid described in Example 1e are added. Addition of 0.48 ml (2.8 mmol) of N,N-ethyldiisopropylamine and 208 mg (0.4 mmol) of benzotriazol-1-yloxytrispyrrolidinophosphoniumhexafluorophosphate (PyBOP) is followed by stirring at RT for 2 days, concentration and partition of the residue between ethyl acetate and sodium bicarbonate solution. The organic phase is washed with water, dried over sodium sulphate, filtered and concentrated.

Yield: 0.74 g (79.8%)

### Elemental Analysis:

| | | | |
|---|---|---|---|
| calculated: | C 64.38 | H 7.23 | N 8.46 |
| found: | C 64.13 | H 6.88 | N 8.56 |

### h) 3-(2-{2-[2-(2-{2-[2-(2-{2-[2-(2,5,2′,4′,6′-Pentamethylbiphenyl-4-yloxy)acetylamino]ethoxy}-ethoxy)ethoxy]ethoxy}ethoxy)ethoxy]ethoxy}ethoxy)propionic acid bis{2-[2,6-bis-(2,6-diaminohexanoylamino)hexanoylamino]ethyl}amide

0.66 g (0.25 mmol) of the completely protected amine described in Example 1g is dissolved in 50 ml of methanol, mixed with 0.5 ml of 2N hydrochloric acid, mixed under nitrogen with 0.2 g of palladium catalyst (10% Pd/C) and stirred under hydrogen for 20 h. The catalyst is then filtered off with suction, the filtrate is concentrated, and the still basic residue is dissolved in water, adjusted to pH 7 with dilute hydrochloric acid, frozen and freeze dried. The resulting colourless powder is employed in the following reaction without further characterization.

Yield: 0.41 g (88% of theory)

### i) Octa-Gd complex amide from the Gd complex of 10-(4-carboxy-1-methyl-2-oxo-3-azabutyl)-1,4,7,10-tetraazacyclododecane-1,4,7-triacetic acid with dendritic octaamine 1h

3.02 g (4.8 mmol) of the gadolinium complex of 10-(4-carboxy-1-methyl-2-oxo-3-azabutyl)-1,4,7,10-tetraazacyclododecane-1,4,7-triacetic acid (Example 1f of EP 0946525) and 0.56 g (4.8 mmol) of N-hydroxysuccinimide are dissolved in 25 ml of dimethyl sulphoxide (DMSO) with heating. After cooling to RT, 1.0 g (4.8 mmol) of N,N′-dicyclohexylcarbodiimide is added, and the mixture is stirred for 60 min. A mixture of 374 mg (0.2 mmol) of the octaamine hydro-chloride described in Example 1h and 0.97 g (9.6 mmol) of triethylamine in 50 ml of DMSO is added to the hydroxysuccinimide active ester solution prepared in situ in this way. After stirring at 50° C. overnight, the volume is made up to about 0.6 l with ethyl acetate and stirred for 24 h, and the precipitate is then filtered off with suction, washed with ethyl acetate and dried in vacuo. The residue is dissolved in water and stirred with 0.5 g of activated carbon for 1 h. The suspension is filtered, the filtrate is ultrafiltered on an AMICON® YM 1 (cut off 1.000 Da) and the retentate is chromatographed with an acetonitrile/water gradient on Lichroprep® RP-18, and the product fractions are freeze dried.

Yield: 0.74 g (55%)

Water content (Karl-Fischer): 4.9%

Elemental Analysis (Based on Anhydrous Substance):

| | | | | |
|---|---|---|---|---|
| calculated: | C 42.70 | H 5.70 | Gd 19.44 | N 12.12 |
| found: | C 42.43 | H 5.88 | Gd 19.07 | N 12.30 |

### EXAMPLE 2

### a) Dimethyl 5-[3-(2-{2-[2-(2-{2-[2-(2-{2-[2-(2,5,2′,4′,6′-pentamethylbiphenyl-4-yloxy)acetyl-amino]ethoxy}ethoxy)ethoxy]ethoxy}ethoxy)ethoxy]ethoxy}ethoxy)propionylamino]-isophthalate

1.37 g (1.9 mmol) of the 3-(2-{2-[2-(2-{2-[2-(2-{2-[2-(2,5,2′,4′,6′-pentamethylbiphenyl-4-yloxy)-acetylamino]ethoxy}ethoxy)ethoxy]ethoxy}ethoxy)ethoxy]ethoxy}ethoxy)propionic acid described in Example 1e are dissolved in 40 ml of THF, and 0.48 g (2.28 mmol) of dimethyl 5-aminoisophthalate (Aldrich) is added. Addition of 1.63 ml (9.5 mmol) of N,N-ethyldiisopropylamine and 1.19 g (2.28 mmol) of PyBOP is followed by stirring at RT overnight. After completion of the reaction, the residue after evaporation in vacuo is chromatographed first with diethyl ether and then with dichloromethane/methanol (19:1) on silica gel.

Yield: 1.0 g (57.6% of theory)

Elemental Analysis:

| | | | |
|---|---|---|---|
| calculated: | C 63.14 | H 7.51 | N 3.07 |
| found: | C 62.94 | H 7.66 | N 3.21 |

### b) 5-[3-(2-{2-[2-(2-{2-[2-(2-{2-[2-(2,5,2′,4′,6′-Pentamethylbiphenyl-4-yloxy)acetylamino]-ethoxy}ethoxy)ethoxy]ethoxy}ethoxy)ethoxy]ethoxy}ethoxy)propionylamino]isophthalic acid

0.91 g (1 mmol) of the dimethyl ester described in Example 2a above is dissolved in 30 ml of THF, mixed with 10 ml (20 mmol) of 2N sodium hydroxide solution and stirred at RT for 3 h. This is followed by dilution with water and adjustment to pH 7 by addition of AMBERLITE® ion exchanger IR 120 (H⁺), and the exchanger is filtered off and the remaining THF is distilled out of the filtrate. The resulting aqueous solution is frozen and freeze dried. The resulting colourless powder is employed in the following reaction without further characterization.

Yield: 0.8 g

### c) Isophthalamide from 5-[3-(2-{2-[2-(2-{2-[2-(2-{2-[2-(2,5,2′,4′,6′-pentamethylbiphenyl-4-yl-oxy)acetylamino]ethoxy}ethoxy)ethoxy]ethoxy}ethoxy)ethoxy]ethoxy}ethoxy)propionyl-amino]isophthalic acid and bis{2-[2,6-bis(2,6-bis(benzyloxycarbonyl)aminohexanoylamino)-hexanoylamino]ethyl}amine

3.89 g (2 mmol) of the protected dendrimer amine described in Example 1f are dissolved in 60 ml of DMF, and 0.80 g (0.9 mmol) of the diacid described in Example 2b is added. Addition of 1.29 g (10 mmol) of N,N-ethyldiisopropylamine and 1.04 g (2 mmol) of PyBOP is followed by stirring at RT overnight. After completion of the reaction, the residue after evaporation in vacuo is chromatographed on silica gel with dichloromethane/methanol (18:2).

Yield: 1.45 g (34% of theory)

Elemental Analysis:

| | | | |
|---|---|---|---|
| calculated: | C 64.37 | H 6.93 | N 9.46 |
| found: | C 64.22 | H 7.03 | N 9.59 |

### d) 5-[3-(2-{2-[2-(2-{2-[2-(2-{2-[2-(2,5,2′,4′,6′-Pentamethylbiphenyl-4-yloxy)acetylamino]-ethoxy}ethoxy)ethoxy]ethoxy}ethoxy)ethoxy]ethoxy}ethoxy)propionylamino]isophthalic acid bis-<N,N-bis{2-[2,6-bis(2,6-diaminohexanoylamino)hexanoylamino]ethyl}->amide

1.4 g (0.3 mmol) of the completely protected amine described in Example 2c are dissolved in 15 ml of glacial acetic acid, mixed with 15 ml of 33% HBr in glacial acetic acid and stirred at RT for 1 h, and the resulting suspension is mixed with 250 ml of diethyl ether, filtered with suction and thoroughly washed with diethyl ether. The residue is dissolved in water and passed over 75 ml of AMBERLITE® ion exchanger IRA 410 (OH—), and the alkaline eluate is frozen and freeze dried. The resulting colourless powder is employed in the following reaction without further characterization.

Yield: 0.7 g (90% of theory)

### e) Hexadeca-Gd complex amide from the Gd complex of 10-(4-carboxy-1-methyl-2-oxo-3-azabutyl)-1,4,7,10-tetraazacyclododecane-1,4,7-triacetic acid with dendritic hexadecaamine 5-[3-(2-{2-[2-(2-{2-[2-(2-{2-[2-(2,5,2′,4′,6′-pentamethylbiphenyl-4-yloxy)acetylamino]-ethoxy}ethoxy)ethoxy]ethoxy}ethoxy)ethoxy]ethoxy}ethoxy)propionylamino]isophthalic acid bis-<N,N-bis{2-[2,6-bis(2,6-diaminohexanoylamino)hexanoylamino]ethyl}->amide

7.55 g (12 mmol) of the gadolinium complex of 10-(4-carboxy-1-methyl-2-oxo-3-azabutyl)-1,4,7,10-tetraazacyclododecane-1,4,7-triacetic acid (Example 1f of EP 0946525) and 1.40 g (12 mmol) of N-hydroxysuccinimide are dissolved in 60 ml of DMSO with heating. After cooling to RT, 2.50 g (12 mmol) of N,N′-dicyclohexylcarbodiimide are added, and the mixture is stirred for 60 min. A mixture of 0.65 g (0.25 mmol) of the hexadecaamine described in Example 2d and 2.43 g (24 mmol) of triethylamine in 60 ml of DMSO are added to the hydroxy-succinimide active ester solution prepared in situ in this way. Stirring at 50° C. overnight and at RT for 3 d is followed by making up the volume to about 0.6 l with ethyl acetate and stirring for 3 h, and the precipitate is then filtered off with suction, washed with ethyl acetate and dried in vacuo. The residue is dissolved in water and stirred with 0.5 g of activated carbon for 1 h. The suspension is filtered, the filtrate is ultrafiltered on an AMICON® YM 1 (cut off 1.000 Da), and the retentate is chromatographed with an acetonitrile/water gradient on Lichroprep® RP-18, and the product fractions are freeze dried.

Yield: 0.62 g (20%)

Water content (Karl-Fischer): 3.5%

Elemental Analysis (Based on the Anhydrous Substance):

| | | | | |
|---|---|---|---|---|
| calculated: | C 41.72 | H 5.52 | Gd 20.32 | N 12.67 |
| found: | C 41.40 | H 5.37 | Gd 19.89 | N 12.81 |

### EXAMPLE 3

### a) Carboxamide from (2,5,2′,4′,6′-pentamethylbiphenyl-4-yloxy)acetic acid and bis{2-[2,6-bis(2,6-bis(benzyloxycarbonyl)aminohexanoylamino)hexanoylamino]ethyl}amine

1.95 g (1 mmol) of bis{2-[2,6-bis(2,6-bis(benzyloxycarbonyl)aminohexanoylamino)hexanoyl-amino]ethyl}amine (Example 1f) are dissolved in 30 ml of DMF, and 298 mg (1 mmol) of the (2,5,2′,4′,6′-pentamethylbiphenyl-4-yloxy)acetic acid described in Example 1c are added. Addition of 1.29 g (10 mmol) of N,N-ethyldiisopropylamine and 676 mg (1.3 mmol) of PyBOP is followed by stirring at RT for 2 days, and concentration, and the residue is partitioned between ethyl acetate and sodium bicarbonate solution. The organic phase is washed with water, dried over sodium sulphate, filtered and concentrated.

Yield: 2.20 g (98.8%)

Elemental Analysis:

| | | | |
|---|---|---|---|
| calculated: | C 66.38 | H 6.93 | N 9.44 |
| found: | C 66.17 | H 6.81 | N 9.65 |

### b) 2,6-Diaminohexanoic acid [5-(2-{{2-[2,6-bis(2,6-diaminohexanoylamino)hexanoyl-amino]ethyl}-[2-(2,5,2′,4′,6′-pentamethylbiphenyl-4-yloxy)acetyl]amino}ethylcarbamoyl)-5-(2,6-diaminohexanoylamino)pentyl]amide

4.23 g (1.9 mmol) of the completely protected amine described in Example 3a are dissolved in 50 ml of glacial acetic acid, mixed with 50 ml of 33% HBr in glacial acetic acid and stirred at RT for 1 h, and the resulting suspension is mixed with 1000 ml of diethyl ether, filtered with suction and thoroughly washed with diethyl ether. The residue is dissolved in water and passed over 75 ml of AMBERLITE® ion exchanger IRA 410 (OH—), and the alkaline eluate is frozen and freeze dried. The resulting colourless powder is employed in the following reaction without further characterization.

Yield: 1.65 g (75.4% of theory)

### c) Octa-Gd complex amide from the Gd complex of 10-(4-carboxy-1-methyl-2-oxo-3-azabutyl)-1,4,7,10-tetraazacyclododecane-1,4,7-triacetic acid with dendritic octaamine 3b

11.32 g (18 mmol) of the gadolinium complex of 10-(4-carboxy-1-methyl-2-oxo-3-azabutyl)-1,4,7,10-tetraazacyclododecane-1,4,7-triacetic acid (Example 1f of EP 0946525) and 2.10 g (18 mmol) of N-hydroxysuccinimide are dissolved in 100 ml of DMSO with heating. After cooling to RT, 3.71 g (18 mmol) of N,N′-dicyclohexylcarbodiimide are added, and the mixture is stirred for 60 min. A mixture of 865 mg (0.75 mmol) of the octaamine described in Example 3b and 4.99 ml (36 mmol) of triethylamine in 100 ml of DMSO is added to the hydroxysuccinimide active ester solution prepared in situ in this way. Stirring at 50° C. overnight is followed by making up the volume to about 1.6 1 with ethyl acetate and stirring for 24 h, and the precipitate is then filtered off with suction, washed with ethyl acetate and dried in vacuo. The residue is dissolved in water and stirred with 2 g of activated carbon for 1 h. The suspension is filtered, the filtrate is ultrafiltered on an AMICON® YM 1 (cut off 1.000 Da), and the retentate is chromato-graphed with an acetonitrile/water gradient on Lichroprep® RP-18, and the product fractions are freeze dried.

Yield: 2.66 g (55%)

Water content (Karl-Fischer): 6.3%

Elemental Analysis (Based on the Anhydrous Substance):

| | | | | |
|---|---|---|---|---|
| calculated: | C 41.92 | H 5.48 | Gd 20.81 | N 12.74 |
| found: | C 41.88 | H 5.31 | Gd 20.22 | N 12.49 |

### EXAMPLE 4

### a) Methyl 11-[2-(2,5,2′,4′,6′-pentamethylbiphenyl-4-yloxy)acetylamino]undecanoate

4.48 g (15 mmol) of (2,5,2′,4′,6′-pentamethylbiphenyl-4-yloxy)acetic acid (Example 1c) and 3.78 g (15 mmol) of methyl 11-aminoundecanoate hydrochloride (Chem. Ber. 94, 2470-2477 (1961)) are taken up in 125 ml of DMF and, after addition of 6.47 ml (37.8 mmol) of N,N-ethyldiisopropylamine and 6.26 g (16.5 mmol) of HBTU, stirred at RT overnight. The resulting crude product is chromatographed on silica gel (dichloromethane/methanol 19:1). The fractions containing the product are combined and evaporated.

Yield: 7 g (94.1% of theory)

Elemental Analysis:

| | | | |
|---|---|---|---|
| calculated: | C 75.11 | H 9.15 | N 2.83 |
| found: | C 75.24 | H 9.02 | N 2.77 |

### b) 11-[2-(2,5,2′,4′,6′-Pentamethylbiphenyl-4-yloxy)acetylamino]undecanoic acid

6.94 g (14 mmol) of the methyl ester described in Example 4a are dissolved in 100 ml of THF, mixed with 35 ml of 2N NaOH and stirred at RT for 20 h. THF is then removed by distillation, the remaining solution is diluted with water, mixed with ethyl acetate and washed several times with 2N hydrochloric acid and finally with half-saturated sodium chloride solution, dried over sodium sulphate, filtered and concentrated. The resulting crude product is chromatographed on silica gel (dichloromethane/methanol 18:2). The fractions containing the product are combined and evaporated.

Yield: 5.1 g (75.6% of theory)

Elemental Analysis:

| | | | |
|---|---|---|---|
| calculated: | C 74.81 | H 9.00 | N 2.91 |
| found: | C 74.63 | H 9.07 | N 2.86 |

### c) Carboxamide from 11-[2-(2,5,2′,4′,6′-pentamethylbiphenyl-4-yloxy)acetylamino]undecanoic acid and bis{2-[2,6-bis-(2,6-bis(benzyloxycarbonyl)aminohexanoylamino)hexanoylamino]-ethyl}amine

3.89 g (2 mmol) of bis{2-[2,6-bis-(2,6-bis(benzyloxycarbonyl)aminohexanoylamino)hexanoyl-amino]ethyl}amine from (Example 1f) are dissolved in 60 ml of DMF, and 0.96 g (2 mmol) of 11-[2-(2,5,2′,4′,6′-pentamethylbiphenyl-4-yloxy)acetylamino]undecanoic acid (Example 4b) is added. Addition of 1.29 g (10 mmol) of N,N-ethyldiisopropylamine and 1.09 g (2.1 mmol) of PyBOP is followed by stirring at RT for 2 days and subsequent concentration. The resulting crude product is adsorbed onto Isolute® HM-N and chromatographed on silica gel (dichloromethane/methanol 18:2). The fractions containing the product are combined and evaporated.

Yield: 4.40 g (91.3%)

Elemental Analysis:

| | | | |
|---|---|---|---|
| calculated: | C 66.81 | H 7.28 | N 9.30 |
| found: | C 66.95 | H 7.42 | N 9.12 |

### d) 11-[2-(2,5,2′,4′,6′-Pentamethylbiphenyl-4-yloxy)acetylamino]undecanoic acid bis{2-[2,6-bis(2,6-diaminohexanoylamino)hexanoylamino]ethyl}amide

2.05 g (0.85 mmol) of the completely protected amine described in Example 4c are dissolved in 25 ml of glacial acetic acid, mixed with 25 ml of 33% HBr in glacial acetic acid and stirred at RT for 1 h, and the resulting suspension is mixed with 500 ml of diethyl ether, filtered with suction and thoroughly washed with diethyl ether. The residue is dissolved in water and passed over 50 ml of AMBERLITE® ion exchanger IRA 410 (OH—), and the alkaline eluate is frozen and freeze dried. The resulting colourless powder is employed in the following reaction without further characterization.

Yield: 0.7 g (61.7% of theory)

### e) Octa-Gd complex amide from the Gd complex of 10-(4-carboxy-1-methyl-2-oxo-3-azabutyl)-1,4,7,10-tetraazacyclododecane-1,4,7-triacetic acid with dendritic octaamine 4d

7.55 g (12 mmol) of the gadolinium complex of 10-(4-carboxy-1-methyl-2-oxo-3-azabutyl)-1,4,7,10-tetraazacyclododecane-1,4,7-triacetic acid (Example 1f of EP 0946525) and 1.4 g (12 mmol) of N-hydroxysuccinimide are dissolved in 70 ml of DMSO with heating. After cooling to RT, 2.47 g (12 mmol) of N,N′-dicyclohexylcarbodiimide are added and the mixture is stirred for 60 min. A mixture of 0.67 g (0.5 mmol) of the octaamine described in Example 4d and 3.33 ml (24 mmol) of triethylamine in 70 ml of DMSO is added to the hydroxysuccinimide active ester solution prepared in situ in this way. Stirring at 50° C. overnight is followed by making up the volume to about 1.4 l with ethyl acetate and stirring for 24 h, and the precipitate is then filtered off with suction, washed with ethyl acetate and dried in vacuo. The residue is dissolved in water and stirred with 2 g of activated carbon for 1 h. The suspension is filtered, the filtrate is ultrafiltered on an AMICON® YM 1 (cut off 1.000 Da), and the retentate is chromatographed with an acetonitrile/water gradient on Lichroprep® RP-18, and the product fractions are freeze dried.

Yield: 2.22 g (65.8%)

Water content (Karl-Fischer): 8.0%

Elemental Analysis (Based on the Anhydrous Substance):

| | | | | |
|---|---|---|---|---|
| calculated: | C 42.80 | H 5.66 | Gd 20.19 | N 12.59 |
| found: | C 42.88 | H 5.48 | Gd 19.82 | N 12.74 |

### EXAMPLE 5

### a) 9H-Fluoren-9-ylmethyl[2-(bis{2-[2,6-bis(2,6-bis(benzyloxycarbonyl)aminohexanoylamino)-hexanoylamino]ethyl}carbamoyl)ethyl]carbamate

### Short name: Fmoc-beta-Ala-N[en₂Lys₆Z₈])₂

1.95 g (1 mmol) of bis{2-[2,6-bis(2,6-bis(benzyloxycarbonyl)aminohexanoylamino)hexanoyl-amino]ethyl}amine (Example 1f) are dissolved in 30 ml of hot DMF. After cooling to room temperature, this solution is added to a solution of 311 mg (1 mmol) of Fmoc-beta-alanine (Fluka Chemie), 206 mg (1 mmol) of N,N′-dicyclohexylcarbodiimide and 320 mg (2 mmol) of 1-hydroxybenzotriazole in 15 ml of DMF. Stirring at RT overnight is followed by making up the volume to 500 ml with diethyl ether and stirring for 5 hours. The precipitated substance is filtered off with suction, washed with diethyl ether and dried in vacuo at 30° C. The resulting crude product is chromatographed on silica gel (dichloromethane/methanol 18:2). The fractions containing the product are combined and evaporated.

Yield: 2.15 g (96% of theory)

Elemental Analysis:

| | | | |
|---|---|---|---|
| calculated: | C 65.46 | H 6.66 | N 10.01 |
| found: | C 65.28 | H 6.77 | N 9.92 |

### b) 2,6-Bis(benzyloxycarbonyl)aminohexanoic acid [5-[2-((3-aminopropionyl)-{2-[2,6-bis(2,6-bis(benzyloxycarbonyl)aminohexanoylamino)hexanoylamino]ethyl}amino)ethylcarbamoyl]-5-(2,6-bis(benzyloxycarbonylaminohexanoylamino)pentyl]amide

### Short name: beta-Ala-N[en₂Lys₆Z₈])₂

1.90 g (0.85 mmol) of the Fmoc compound described in the preceding example are suspended in 200 ml of methanol, mixed with 42.5 ml of piperidine and stirred at RT overnight. The undissolved substance is filtered off with suction, washed with methanol, then washed with diethyl ether and dried in vacuo at 30° C. The resulting colourless powder is employed in the following reaction without further characterization.

Yield: 1.3 g (75.9% of theory)

### c) Dimethyl 5-[2-(2,5,2′,4′,6′-pentamethylbiphenyl-4-yloxy)acetylamino]isophthalate

7.46 g (25 mmol) of (2,5,2′,4′,6′-pentamethylbiphenyl-4-yloxy)acetic acid (Example 1c) are dissolved in 125 ml of dichloromethane and mixed with 0.5 ml of DMF. 3.49 g (2.38 ml, 27.5 mmol) of oxalyl chloride are added dropwise to the solution, and the reaction mixture is first stirred under reflux for 90 minutes and then cooled to 0° C. Subsequently, 5.23 g (25 mmol) of dimethyl 5-aminoisophthalate and 5.57 g (55 mmol) of triethylamine in 200 ml of dichloro-methane are added. The mixture is stirred in ice for 2 h and at RT overnight. The organic phase is washed successively with sodium bicarbonate solution, 2N hydrochloric acid and saturated NaCl solution, dried over sodium sulphate, filtered and concentrated. The residue is adsorbed on silica gel and chromatographed with diisoproypl ether/diethyl ether (4:1).

Yield: 8.7 g (71.1% of theory)

Elemental Analysis:

| | | | |
|---|---|---|---|
| calculated: | C 71.15 | H 6.38 | N 2.86 |
| found: | C 70.83 | H 6.24 | N 2.91 |

### d) 5-[2-(2,5,2′,4′,6′-Pentamethylbiphenyl-4-yloxy)acetylamino]isophthalic acid

7.34 g (15 mmol) of the dimethyl ester described in Example 5c are dissolved in 100 ml of THF, mixed with 30 ml (60 mmol) of 2N sodium hydroxide solution, stirred at RT for 5 h and then adjusted to pH 7 with 2N hydrochloric acid. The THF is concentrated in vacuo, and the remaining aqueous solution is mixed with ethyl acetate. The organic phase is washed with 2N hydrochloric acid and saturated NaCl solution, dried over sodium sulphate, filtered and concentrated. The residue is suspended in 250 ml of diisopropyl ether and stirred overnight, and the substance is filtered off with suction, washed with diisopropyl ether and dried in vacuo at 40° C.

Yield: 5.1 g (73.7% of theory)

Elemental Analysis:

| | | | |
|---|---|---|---|
| calculated: | C 70.27 | H 5.90 | N 3.03 |
| found: | C 70.32 | H 5.88 | N 3.16 |

### e) N,N′-Bis{2-[2-(2-{2-[2-(2-{2-[2-(2-tert-butoxycarbonylethoxy)ethoxy]ethoxy}ethoxy)-ethoxy]ethoxy}ethoxy)ethoxy]ethyl}-5-[2-(2,5,2′,4′,6′-pentamethylbiphenyl-4-yloxy)-acetylamino]isophthalamide

### (Me₅biphenylbiphenyl-CO—NHC₆H₃(CONH—PEG8-COOtBu)₂

0.46 g (1 mmol) of the diacid described in Example 5d is dissolved with 1.00 g (2 mmol) of amino-dPEG8 tert-butyl ester (QUANTA Biodesign, Powell, Ohio, USA, Product No. 10271) in 20 ml of THF, mixed with 1.02 ml (2.1 mmol) of N,N-ethyldiisopropylamine and 0.80 g (2.1 mmol) of HBTU and stirred overnight and concentrated. The residue is chromatographed on silica gel (dichloromethane/methanol 19:1).

Yield: 1.2 g (84.5% of theory)

Elemental Analysis:

| | | | |
|---|---|---|---|
| calculated: | C 61.71 | H 8.30 | N 2.96 |
| found: | C 61.44 | H 8.38 | N 3.06 |

### f) N,N′-Bis{2-[2-(2-{2-[2-(2-{2-[2-(2-carboxyethoxy)ethoxy]ethoxy}-ethoxy)ethoxy]ethyl}-5-[2-(2,5,2′,4′,6′-pentamethylbiphenyl-4-yloxy)acetylamino]-isophthalamide

### (Me₅biphenylbiphenyl-CO—NHC₆H₃(CONH—PEG8-COOH)₂

1.14 g (0.8 mmol) of the bis-tert-butyl ester described in Example 5e are dissolved in 60 ml of HCl/diethyl ether (4.5 M), mixed with 20 ml of THF and stirred at RT overnight, concentrated and then distilled with diethyl ether and THF several times. The residue is chromatographed with acetonitrile/water gradient on Lichroprep® RP-18, and the product fractions are freeze dried. The resulting colourless powder is employed in the following reaction without further characterization.

Yield: 0.27 g

### g) Bisamide from diacid 5f und beta-Alanyl-Z8 dendrimer 5b

### (Me₅biphenylbiphenyl-CO—NHC₆H₃(CONH—PEG8-CO-beta-Ala-N[en₂Lys₆Z₈])₂

0.26 g (0.2 mmol) of the diacid described in Example 5f and 1.01 g (0.5 mmol) of the amine described in Example 5b are dissolved in 50 ml of DMF. Addition of 0.41 g (3.2 mmol) of N,N-ethyldiisopropylamine and 0.26 g (0.5 mmol) of PyBOP is followed by stirring at RT overnight. After completion of the reaction, the residue after evaporation in vacuo is taken up in dichloromethane and stirred overnight and the undissolved substance is filtered off, washed with dichloromethane and dried in vacuo. The resulting colourless powder is employed in the following reaction without further characterization.

Yield: 1.10 g

### h) Deprotected 16-amin from the completely protected benzyloxycarbonyl dendrimer 5h

### (Me₅biphenylbiphenyl-CO—NHC₆H₃(CONH—PEG8-CO-beta-Ala-N[en₂Lys₆H₈])₂

1.06 g (0.2 mmol) of the completely protected amine described in Example 5g are dissolved in 15 ml of glacial acetic acid, mixed with 15 ml of 33% HBr in glacial acetic acid and stirred at RT for 1 h, and the resulting suspension is mixed with 250 ml of diethyl ether, filtered with suction and thoroughly washed with diethyl ether. The residue is dissolved in water and passed over about 40 ml of AMBERLITE® ion exchanger IRA 410 (OH—), and the alkaline eluate is frozen and freeze dried. The resulting colourless powder is employed in the following reaction without further characterization.

Yield: 0.65 g

### i) Hexadeca-Gd complex amide from the Gd complex of 10-(4-carboxy-1-methyl-2-oxo-3-azabutyl)-1,4,7,10-tetraazacyclododecane-1,4,7-triacetic acid with dendritic hexadecaamine 5h

### (Me₅biphenylbiphenyl-CO—NHC₆H₃(CONH—PEG8-CO-beta-Ala-N[en₂Lys₆Gd₈])₂

6.04 g (9.6 mmol) of the gadolinium complex of 10-(4-carboxy-1-methyl-2-oxo-3-azabutyl)-1,4,7,10-tetraazacyclododecane-1,4,7-triacetic acid (Example 1f of EP 0946525) and 1.12 g (9.6 mmol) of N-hydroxysuccinimide are dissolved in 50 ml of DMSO with heating. After cooling to RT, 2.0 g (9.6 mmol) of N,N′-dicyclohexylcarbodiimide are added, and the mixture is stirred for 60 min. A mixture of 631 mg (0.2 mmol) of the hexadecaamine described in Example 5h and 1.94 g (19.2 mmol) of triethylamine in 50 ml of DMSO is added to the hydroxy-succinimide active ester solution prepared in situ in this way. Stirring at 50° C. overnight and at RT for 3 d is followed by making up the volume to about 0.6 l with ethyl acetate and stirring for 3 h, and the precipitate is then filtered off with suction, washed with ethyl acetate and dried in vacuo. The residue is dissolved in water and stirred with 0.5 g of activated carbon for 1 h. The suspension is filtered, the filtrate is ultrafiltered on an AMICON® YM 1 (cut off 1.000 Da), and the retentate is chromatographed with a water/acetonitrile gradient on Lichroprep® RP-18, and the product fractions are freeze dried.

Yield: 0.6 g (23%)

Water content (Karl-Fischer): 8.5%

Elemental Analysis (Based on the Anhydrous Substance):

| | | | | |
|---|---|---|---|---|
| calculated: | C 42.21 | H 5.64 | Gd 19.43 | N 12.44 |
| found: | C 41.99 | H 5.84 | Gd 19.04 | N 12.86 |

### EXAMPLE 6

### a) Dimethyl 5-[(biphenyl-4-carbonyl)amino]isophthalate

10.36 g (47.8 mmol) of 4-biphenylcarbonyl chloride (Aldrich) are dissolved in 300 ml of dichloromethane, and 10.0 g (47.8 mmol) of dimethyl 5-aminoisophthalate and 8.71 ml (50 mmol) of N,N-ethyldiisopropylamine are added. The reaction mixture is stirred at RT for 5 h and then washed several times with sodium bicarbonate solution, dilute hydrochloric acid and saturated sodium chloride solution. The organic phase is dried over sodium sulphate, filtered and concentrated.

Yield: 15.1 g (81.1% of theory)

Elemental Analysis:

| | | | |
|---|---|---|---|
| calculated: | C 70.94 | H 4.92 | N 3.60 |
| found: | C 70.72 | H 5.05 | N 3.72 |

### b) 5-[(Biphenyl-4-carbonyl)amino]isophthalic acid

5.84 g (15 mmol) of the dimethyl ester described in Example 6a are dissolved in 100 ml of THF, mixed with 30 ml (60 mmol) of 2N sodium hydroxide solution, stirred at RT for 5 h and then adjusted to pH 7 with 2N hydrochloric acid. The THF is concentrated in vacuo and the remaining aqueous solution is mixed with ethyl acetate. The organic phase is washed with 2N hydrochloric acid and saturated NaCl solution, dried over sodium sulphate, filtered and concentrated. The residue is suspended in 250 ml of diisopropyl ether and stirred overnight, and the substance is filtered off with suction, washed with diisopropyl ether and dried in vacuo at 40° C.

Yield: 3.8 g (70.7% of theory)

Elemental Analysis:

| | | | |
|---|---|---|---|
| calculated: | C 69.80 | H 4.18 | N 3.88 |
| found: | C 69.78 | H 4.29 | N 3.71 |

### c) N,N′-Bis{2-[2-(2-{2-[2-(2-{2-[2-(2-tert-butoxycarbonylethoxy)ethoxy]ethoxy}ethoxy-ethoxy]ethoxy}ethoxy)ethoxy]ethyl}-5-[(biphenyl-4-carbonyl)amino]isophthalamide

### (Biphenyl-CO—NHC₆H₃(CONH—PEG8-COOtBu)₂

361 mg (1 mmol) of the diacid described in Example 6b are dissolved with 1.00 g (2 mmol) of amino-dPEG8 tert-butyl ester (QUANTA Biodesign, Powell, Ohio, USA, Product No. 10271) in 20 ml of THF, mixed with 1.02 ml (2.1 mmol) of N,N-ethyldiisopropylamine and 0.80 g (2.1 mmol) of HBTU and stirred overnight and concentrated. The residue is chromatographed on silica gel (dichloromethane/methanol 9:1).

Yield: 1.15 g (87.0% of theory)

Elemental Analysis:

| | | | |
|---|---|---|---|
| calculated: | C 60.94 | H 8.01 | N 3.18 |
| found: | C 60.69 | H 7.88 | N 3.29 |

### d) N,N′-Bis{2-[2-(2-{2-[2-(2-{2-[2-(2-carboxyethoxy)ethoxy]ethoxy}ethoxy)ethoxy]ethoxy}ethoxy)ethoxy]ethyl}-5-[(biphenyl-4-carbonyl)amino]isophthalamide

### (Biphenyl-CO—NHC₆H₃(CONH—PEG₈-COOH)₂

1.06 g (0.8 mmol) of the bis-tert-butyl ester described in Example 6c are dissolved in 60 ml of HCl/diethyl ether (4.5 M), mixed with 20 ml of THF and stirred at RT overnight, concentrated and then distilled with diethyl ether and THF several times. The residue is chromatographed with acetonitrile/water gradient on Lichroprep® RP-18, and the product fractions are freeze dried. The resulting colourless powder is employed in the following reaction without further characterization.

Yield: 0.32 g

### e) Bisamide from diacid 6d and Z8 dendrimer 1f

### (Biphenyl-CO—NHC₆H₃ (CONH—PEG8-CO—N[en₂Lys₆Z₈])₂

0.24 g (0.2 mmol) of the diacid described in Example 6d and 0.97 g (0.5 mmol) of the amine described in Example 1f are dissolved in 50 ml of DMF. Addition of 0.41 g (3.2 mmol) of N,N-ethyldiisopropylamine and 0.26 g (0.5 mmol) of PyBOP is followed by stirring at RT overnight. After completion of the reaction, the residue after evaporation in vacuo is taken up in dichloromethane and stirred overnight and the undissolved substance is filtered off, washed with dichloromethane and dried in vacuo. The resulting colourless powder is employed in the following reaction without further characterization.

Yield: 0.99 g

### f) Deprotected 16-amine from the Completely Protected Benzyloxycarbonyl Dendrimer 6e

### (Biphenyl-CO—NHC₆H₃ (CONH—PEG8-CO—N[en₂Lys₆H₈])₂

1.01 g (0.2 mmol) of the completely protected amine described in Example 6e are dissolved in 15 ml of glacial acetic acid, mixed with 15 ml of 33% HBr in glacial acetic acid and stirred at RT for 1 h, and the resulting suspension is mixed with 250 ml of diethyl ether, filtered off with suction and thoroughly washed with diethyl ether. The residue is dissolved in water and passed over about 40 ml of AMBERLITE® ion exchanger IRA 410 (OH—), and the alkaline eluate is frozen and freeze dried. The resulting colourless powder is employed in the following reaction without further characterization.

Yield: 0.58 g

### g) Hexadeca-Gd complex amide from the Gd complex of 10-(4-carboxy-1-methyl-2-oxo-3-azabutyl)-1,4,7,10-tetraazacyclododecane-1,4,7-triacetic acid with dendritic hexadecaamine 6f

### (Biphenyl-CO—NHC₆H₃(CONH—PEG8-CO—N[en₂Lys₆Gd₈])₂

6.04 g (9.6 mmol) of the gadolinium complex of 10-(4-carboxy-1-methyl-2-oxo-3-azabutyl)-1,4,7,10-tetraazacyclododecane-1,4,7-triacetic acid (Example 1f of EP 0946525) and 1.12 g (9.6 mmol) of N-hydroxysuccinimide are dissolved in 50 ml of DMSO with heating. After cooling to RT, 2.0 g (9.6 mmol) of N,N′-dicyclohexylcarbodiimide are added, and the mixture is stirred for 60 min. A mixture of 583 mg (0.2 mmol) of the hexadecaamine described in Example 6f and 1.94 g (19.2 mmol) of triethylamine in 50 ml of DMSO is added to the hydroxy-succinimide active ester solution prepared in situ in this way. Stirring at 50° C. overnight and at RT for 3 d is followed by making up the volume to about 0.6 l with ethyl acetate and stirring for 3 h, and the precipitate is then filtered off with suction, washed with ethyl acetate and dried in vacuo. The residue is dissolved in water and stirred with 0.5 g of activated carbon for 1 h. The suspension is filtered, the filtrate is ultrafiltered on an AMICON® YM 1 (cut off 1.000 Da), and the retentate is chromatographed with a water/acetonitrile gradient on Lichroprep® RP-18, and the product fractions are freeze dried.

Yield: 0.70 g (25%)

Water content (Karl-Fischer): 10.0%

Elemental Analysis (Based on the Anhydrous Substance):

| | | | | |
|---|---|---|---|---|
| calculated: | C 41.88 | H 5.58 | Gd 19.80 | N 12.46 |
| found: | C 41.43 | H 5.77 | Gd 19.21 | N 12.72 |

### EXAMPLE 7

### a) Methyl 12-[tert-butoxy-(2,5,2′,4′,6′-pentamethylbiphenyl-4-yloxy)phosphoryloxy]-dodecanoate

1.05 g (4.36 mmol) of the 2,5,2′,4′,6′-pentamethylbiphenyl-4-ol described in Example 1a are stirred in 10 ml of acetonitrile and with 1.72 g (5.66 mmol) of tert-butyl tetraisopropylphosphordiamidite (Aldrich) and 0.97 g (5.66 mmol) of diisopropylammoniumtetrazolide (Chem-Impex International, Inc.) at RT under nitrogen for 5h. The suspension is concentrated in vacuo, the residue is stirred with 20 ml of diethyl ether, insolubles are filtered off, and the solution is concentrated. The resulting crude product is immediately chromatographed on silica gel (hexane/ethyl acetate 9:1). The fractions containing the product are combined and evaporated.

Yield: 1.16 g.

The phosphoramidite prepared in this way is dissolved with 0.50 g (2.17 mmol) of methyl 12-hydroxydodecanoate in 15 ml of dichloromethane in dried glass apparatus, at 0° C. 3 g of 4 \acute{Å} molecule sieves and 8.6 ml of a 3% strength solution of tetrazole in acetonitrile are added, and the mixture is stirred at 0° C. for 1 h and at RT for 3 h. Then 0.72 ml of 80% strength t-butyl hydroperoxide is added, and the mixture is stirred at RT overnight. It is filtered and the solution is concentrated. The resulting crude product is chromatographed on silica gel (diethyl ether/dichloromethane 1:1) and the fractions containing the product are combined and evaporated. The resulting colourless product is employed in the following reaction without further characterization.

Yield: 1.05 g.

### b) Sodium 12-[hydroxy-(2,5,2′,4′,6′-pentamethylbiphenyl-4-yloxy)phosphoryloxy]dodecanoate

### (Me₅biphenylbiphenyl-O—PO(ONa)OC₁₁H₂₂COOH)

0.33 g (0.62 mmol) of the ester described in Example 7a is dissolved in 30 ml of methanol and mixed with 5 ml (10 mmol) of 2N sodium hydroxide solution and stirred at RT overnight. The pH is then adjusted to 7 with dilute hydrochloric acid, and the solution is evaporated to dryness. The crude product is dissolved in ethyl acetate, and the organic phase is washed with citric acid solution, washed with water until neutral and dried over sodium sulphate.

Yield: 250 mg (74.5% of theory)

Elemental Analysis:

| | | | | |
|---|---|---|---|---|
| calculated: | C 64.43 | H 7.83 | Na 4.25 | P 5.73 |
| found: | C 64.31 | H 8.04 | Na 2.80 | P 5.82 |

### c) Carboxamide from sodium 12-[hydroxy-(2,5,2′,4′,6′-pentamethylbiphenyl-4-yloxy)-phosphoryloxy]dodecanoate and bis{2-[2,6-bis(2,6-bis(benzyloxycarbonyl)amino-hexanoylamino)hexanoylamino]ethyl}amine

### (Me₅biphenylbiphenyl-O—PO(ONa)OC₁₁H₂₂CO N[en₂Lys₆Z₈])₂)

3.6 g (1.85 mmol) of bis{2-[2,6-bis(2,6-bis(benzyloxycarbonyl)aminohexanoylamino)-hexanoylamino]ethyl}amine (Example 1f) are dissolved in 50 ml of DMF, and 0.96 g (1.85 mmol) of the dodecanoic acid derivative described in Example 7b is added. Addition of 1.0 ml (5.84 mmol) of N,N-ethyldiisopropylamine and 0.96 g (1.84 mmol) of PyBOP is followed by stirring at RT for 2 days, concentration and partition of the residue between ethyl acetate and sodium bicarbonate solution. The organic phase is washed with water, dried over sodium sulphate, filtered and concentrated.

Yield: 3.04 g (67.1%)

Elemental Analysis:

| | | | | | |
|---|---|---|---|---|---|
| calculated: | C 64.73 | H 7.07 | N 8.51 | Na 0.93 | P 1.26 |
| found: | C 65.00 | H 7.20 | N 8.68 | Na 0.68 | P 1.09 |

### d) 11-(Bis{2-[(S)-2,6-bis((S)-2,6-diaminohexanoylamino)hexanoylamino]ethyl}carbamoyl)-undecyl 2,5,2′,4′,6′-pentamethylbiphenyl-4-yl phosphate

### (Me₅biphenylbiphenyl-O—PO₃C₁₁H₂₂CO N[en₂Lys₆H₈])₂)

3.0 g (1.23 mmol) of the completely protected amine described in Example 7c are dissolved in 300 ml of methanol, mixed with 0.5 ml of conc. hydrochloric acid and, under nitrogen, 1.5 g of palladium catalyst (10% Pd/C) are added, and the mixture is stirred under hydrogen for 20 h. The catalyst is then filtered off with suction, the filtrate is concentrated, and the still base residue is dissolved in water, adjusted to pH 7 with dilute hydrochloric acid, frozen and freeze dried. The resulting colourless powder is employed in the following reaction without further characterization.

Yield: 2.0 g

### e) Octa-Gd complex amide from the Gd complex of 10-(4-carboxy-1-methyl-2-oxo-3-azabutyl)-1,4,7,10-tetraazacyclododecane-1,4,7-triacetic acid with dendritic octaamine 7d

3.02 g (4.8 mmol) of the gadolinium complex of 10-(4-carboxy-1-methyl-2-oxo-3-azabutyl)-1,4,7,10-tetraazacyclododecane-1,4,7-triacetic acid (Example 1f of EP 0946525) and 0.56 g (4.8 mmol) of N-hydroxysuccinimide are dissolved in 25 ml of dimethyl sulphoxide (DMSO) with heating. After cooling to RT, 1.0 g (4.8 mmol) of N,N′-dicyclohexylcarbodiimide is added, and the mixture is stirred for 60 min. A mixture of 325 mg (0.2 mmol) of the octaamine hydrochloride described in Example 7d and 0.97 g (9.6 mmol) of triethylamine in 50 ml of DMSO is added to the hydroxysuccinimide active ester solution prepared in situ in this way. Stirring at 50° C. overnight is followed by making up the volume to about 0.6 l with ethyl acetate and stirring for 24 h, and the precipitate is then filtered off with suction, washed with ethyl acetate and dried in vacuo. The residue is dissolved in water and stirred with 0.5 g of activated carbon for 1 h. The suspension is filtered, the filtrate is ultrafiltered on an AMICON® YM 1 (cut off 1.000 Da), and the retentate is chromatographed with an acetonitrile/water gradient on Lichroprep® RP-18, and the product fractions are freeze dried.

Yield: 132 mg (10%)

Water content (Karl-Fischer): 5.1%

Elemental Analysis (Based on the Anhydrous Substance):

| | | | | | | |
|---|---|---|---|---|---|---|
| calculated: | C 42.21 | H 5.59 | Gd 20.00 | N 12.25 | Na 0.37 | P 0.49 |
| found: | C 42.09 | H 5.68 | Gd 19.46 | N 12.41 | Na 0.41 | P 0.30 |

### EXAMPLE 8

### a) N-(4-Iodophenyl)-2-(2,5,2′,4′,6′-pentamethylbiphenyl-4-yloxy)acetamide

### (Me₅biphenyl-NH—C₆H₄—I)

11.94 g (40 mmol) of the acid described in Example 1c and 8.76 g (40 mmol) of 4-iodoaniline (Aldrich) are dissolved in 400 ml of DMF, mixed with 15.51 g (120 mmol) of N,N-ethyl-diisopropylamine and 22.90 g (44 mmol) of PyBOP, stirred at RT for 2 days and then concentrated in vacuo. The residue is chromatographed on silica gel (hexane/ethyl acetate gradient 98/2-80/20). The fractions containing the product are combined and evaporated.

Yield: 14.7 g (73.6% of theory)

Elemental Analysis:

| | | | | |
|---|---|---|---|---|
| calculated: | C 60.13 | H 5.25 | I 25.41 | N 2.80 |
| found: | C 59.97 | H 5.36 | I 24.88 | N 2.67 |

### b) 2-(2,5,2′,4′,6′-Pentamethylbiphenyl-4-yloxy)-N-(4-trimethylsilanylethynylphenyl)acetamide

### (Me₅biphenyl-NH—C₆H₄—CC—SiMe₃)

3 g (6 mmol) of the iodine compound described in Example 8a are taken up in 30 ml of diethylamine and, after flushing with nitrogen, 1.13 ml (8 mmol) of trimethylsilylacetylene (Fluka) are added. Addition of 84 mg (0.12 mmol) of bis(triphenylphosphine)palladium(II) chloride and 11 mg (0.06 mmol) of copper(I) iodide is followed by stirring at RT overnight, concentration and chromatography of the residue on silica gel (hexane/ethyl acetate gradient 98/2-80/20). The fractions containing the product are combined and employed in the following reaction without further characterization.

Yield: 2.15 g.

### c) N-(4-Ethynylphenyl)-2-(2,5,2′,4′,6′-pentamethylbiphenyl-4-yloxy)acetamide

### (Me₅biphenyl-NH—C₆H₄—CCH)

1.41 g (3 mmol) of the trimethylsilyl compound described in Example 8b are suspended in 60 ml of methanol, mixed with 4.5 ml (4.5 mmol) of 1N aqueous potassium hydroxide solution and stirred at RT overnight. The reaction mixture is concentrated in vacuo and the residue is partitioned between ethyl acetate and water, the organic phase is washed with saturated NaCl solution, dried over sodium sulphate and filtered, and the filtrate is concentrated. The resulting crude product is chromatographed on silica gel (dichloromethane/ethyl acetate gradient 98/2-80/20). The fractions containing the product are combined and evaporated.

Yield: 0.95 g (79.7% of theory)

Elemental Analysis:

| | | | |
|---|---|---|---|
| calculated: | C 81.58 | H 6.85 | N 3.52 |
| found: | C 81.31 | H 6.98 | N 3.30 |

### d) 4-{4-[2-(2,5,2′,4′,6′-Pentamethylbiphenyl-4-yloxy)acetylamino]phenylethynyl}benzoic acid

### Me₅biphenyl-NH—C₆H₄—CC—C₆H₄COOH

358 mg (0.9 mmol) of the acetylene compound described in Example 8c are taken up with 223 mg (0.9 mmol) of 4-iodobenzoic acid in 9 ml of diethylamine and, under nitrogen, 15 mg of bis(triphenylphosphine)palladium(II) chloride and 3 mg of copper(I) iodide are added at RT. The resulting suspension is diluted after 90 min with 10 ml of dichloromethane and concentrated after a total of 2.5 h. The residue is partitioned between dichloromethane and aqueous citric acid solution, the organic phase is washed with saturated NaCl solution, dried over sodium sulphate and filtered, and the filtrate is concentrated. The resulting crude product is chromatographed on silica gel (dichloromethane/methanol gradient). The fractions containing the product are combined and evaporated.

Yield: 370 mg (79.4% of theory)

Elemental Analysis:

| | | | |
|---|---|---|---|
| calculated: | C 78.89 | H 6.04 | N 2.71 |
| found: | C 78.60 | H 6.21 | N 2.88 |

### e) N,N-bis{2-[2,6-bis(2,6-bis(benzyloxycarbonyl)aminohexanoylamino)hexanoylamino]ethyl}-4-{4-[2-(2,5,2′,4′,6′-pentamethylbiphenyl-4-yloxy)acetylamino]phenylethynyl}benzamide

### Me₅biphenyl-NH—C₆H₄—CC—C₆H₄CO N[en₂Lys₆Zs])₂

311 mg (0.6 mmol) of the acid described in Example 8d are taken up in 15 ml of DMF, and 1.17 g (0.6 mmol) of the Z8 compound described in Example 1f are added. After addition of 0.51 ml (3 mmol) of N,N-ethyldiisopropylamine and 0.34 g (0.66 mmol) of PyBOP, the mixture is reacted in a microwave at 120° C. for 15 min. The reaction mixture is concentrated and the residue is chromatographed on silica gel (dichloromethane/methanol gradient). The fractions containing the product are combined and evaporated.

Yield: 1.05 g (71.6% of theory)

Elemental Analysis:

| | | | |
|---|---|---|---|
| calculated: | C 67.80 | H 6.68 | N 9.17 |
| found: | C 67.53 | H 6.77 | N 9.34 |

### f) N,N-bis{2-[2,6-bis(2,6-diaminohexanoylamino)hexanoylamino]ethyl}-4-{4-[2-pentamethylbiphenyl-4-yloxy)acetylamino]phenylethynyl}benzamide

### Me₅biphenyl-NH—C₆H₄—CC—C₆H₄CO N[en₂Lys₆H₈])₂

0.49 g (0.2 mmol) of the Z8 compound described in Example 8e is dissolved in 10 ml of glacial acetic acid, mixed with 10 ml of 33% HBr in glacial acetic acid, stirred at RT for 1 h and made up to 200 ml with diethyl ether. The mixture is stirred for 2 h, and the precipitate is filtered off with suction, washed with diethyl ether and dried in vacuo. The resulting colourless powder is employed in the following reaction without further characterization.

Yield: 0.41 g

### g) Octa-Gd complex amide form the Gd complex of 10-(4-carboxy-1-methyl-2-oxo-3-azabutyl)-1,4,7,10-tetraazacyclododecane-1,4,7-triacetic acid with dendritic octaamine 9f

### Me₅biphenyl-NH—C₆H₄—CC—C₆H₄CO N[en₂Lys₆Gd₈])₂

3.02 g (4.8 mmol) of the gadolinium complex of 10-(4-carboxy-1-methyl-2-oxo-3-azabutyl)-1,4,7,10-tetraazacyclododecane-1,4,7-triacetic acid (Example 1f of EP 0946525) and 0.56 g (4.8 mmol) of N-hydroxysuccinimide are dissolved in 25 ml of dimethyl sulphoxide (DMSO) with heating. After cooling to RT, 1.0 g (4.8 mmol) of N,N′-dicyclohexylcarbodiimide is added, and the mixture is stirred for 60 min. A mixture of 0.2 mmol of the octaamine hydrobromide described in Example 8f and 0.97 g (9.6 mmol) of triethylamine in 60 ml of DMSO is added to the hydroxysuccinimide active ester solution prepared in situ in this way. Stirring at 50° C. overnight is followed by making up the volume to about 0.6 l with ethyl acetate and stirring for 24 h, and the precipitate is then filtered off with suction, washed with ethyl acetate and dried in vacuo. The residue is dissolved in water and stirred with 0.5 g of activated carbon for 1 h. The suspension is filtered, the filtrate is ultrafiltered on an AMICON® YM 1 (cut off 1.000 Da), and the retentate is chromatographed with an acetonitrile/water gradient on Lichroprep® RP-18, and the product fractions are freeze dried.

Yield: 0.55 g (41.0% of theory)

Water content (Karl-Fischer): 6.5%

Elemental Analysis (Based on the Anhydrous Substance):

| | | | | |
|---|---|---|---|---|
| calculated: | C 43.32 | H 5.44 | Gd 20.08 | N 12.52 |
| found: | C 42.96 | H 5.68 | Gd 19.57 | N 12.21 |

### EXAMPLE 9

### a) Cyclohexylhydroxyacetylglycine benzyl ester

50 g (148 mmol) of glycine benzyl ester toluene-4-sulphate (Aldrich) are partitioned between 500 ml of ethyl acetate and 250 ml of saturated sodium carbonate solution. The organic phase is washed with water until neutral, dried over sodium sulphate, filtered and concentrated in vacuo.

Yield: 16.7 g of pale yellowish oil.

Then 13.32 g (84.2 mmol) of cyclohexylhydroxyacetic acid (Journal of the American Chemical Society 103, 1566 (1981)) are dissolved in 100 ml of DMF and, after addition of hydroxybenzotriazole, 16.7 g (101 mmol) of the liberated glycine benzyl ester are added. After 30 min at 0° C., a solution of N,N′-dicyclohexylcarbodiimide in 50 ml of DMF is added, and the mixture is stirred at 0° C. for 30 min and at RT overnight. The precipitated urea is then filtered off, and the filtrate is concentrated in vacuo. The residue is chromatographed on silica gel (hexane/ethyl acetate gradient). The fractions containing the product are combined and evaporated.

Yield: 7.04 g (27.4% of theory)

Elemental Analysis:

| | | | |
|---|---|---|---|
| calculated: | C 66.86 | H 7.59 | N 4.59 |
| found: | C 66.52 | H 7.78 | N 4.50 |

### b) (2-Cyclohexyl-2-trifluoromethanesulphonyloxyacetyl)glycine benzyl ester

A solution of 6.45 g (21.12 mmol) of the alcohol described in Example 9a and 2,6-dimethylpyridine in 30 ml of dichloromethane is slowly added dropwise to a solution of 6.56 g (23.23 mmol) of trifluoromethanesulphonic anhydride in 50 ml of dichloromethane at −60° C. After 2 h at −60° C., the mixture is allowed to warm to −5° C., 100 ml of ice-water are added, and the phases are separated. The organic phase is washed once again with ice-water, dried over magnesium sulphate and concentrated in vacuo. The resulting viscous oil is employed in the following reaction without further characterization.

Yield: 9.0 g.

### c) 10-(4-Benzyloxycarbonyl-1-cyclohexyl-2-oxo-3-azabutyl)-1,4,7,10-tetraazacyclododecane-1,4,7-tris(acetic acid tert-butyl ester), sodium bromide complex

7.44 g (43.21 mmol) of cyclen are dissolved in 80 ml of chloroform, and 9.0 g (20.57 mmol) of the triflate described in Example 9b in 20 ml of chloroform are added. After stirring at RT overnight, the organic phase is washed several times with 150 ml of water each time and then dried over magnesium sulphate and filtered, and the filtrate is concentrated. The residue is dissolved in 70 ml of acetonitrile, and 7.5 g (70.8 mmol) of sodium carbonate are added. 13.82 g (70.85 mmol) of tert-butyl acetate are added dropwise, and the reaction mixture is stirred at 60° C. for 6 h and at RT overnight. Solid is filtered off and the filtrate is evaporated to dryness. The resulting crude product is chromatographed on silica gel (ethyl acetate/ethanol gradient 20/1-1/1). The fractions containing the product are combined and evaporated. The resulting oil is employed in the following reaction without further characterization.

Yield: 2.8 g (21.9% of theory)

### d) 10-(4-Carboxy-1-cyclohexyl-2-oxo-3-azabutyl)-1,4,7,10-tetraazacyclododecane-1,4,7-triacetic acid, sodium bromide complex

1.0 g (1.25 mmol) of the ester described in Example 9c is dissolved in 20 ml of methanol, mixed with a solution of 1.0 g (25 mmol) of NaOH in 10 ml of water and heated under reflux for 4 h and stirred at RT overnight. The residue after subsequent concentration is taken up in water and adjusted to pH 3 by adding AMBERLITE® ion exchanger IR 120 (H⁺), and the exchanger is filtered off and the solution is freeze dried. The resulting colourless powder is employed in the following reaction without further characterization.

Yield: 0.7 g (quantitative)

### e) Gadolinium complex of 10-(4-carboxy-1-cyclohexyl-2-oxo-3-azabutyl)-1,4,7,10-tetra-azacyclododecane-1,4,7-triacetic acid

6.52 g (12 mmol) of the complexing agent acid described in Example 9d in 100 ml of water are adjusted to pH 3 with dilute hydrochloric acid and, after addition of 2.17 g (6 mmol) of gadolinium oxide, stirred at 80° C. for 30 min. After cooling to RT, the pH is adjusted to 7 and the solution is concentrated in vacuo. The residue is chromatographed with an acetonitrile/water gradient on Lichroprep® RP-18, and the product fractions are freeze dried.

Yield: 5.77 g (63.3%)

Water content (Karl-Fischer): 8.1%

Elemental Analysis (Based on the Anhydrous Substance):

| | | | | |
|---|---|---|---|---|
| calculated: | C 41.31 | H 5.49 | Gd 22.53 | N 10.04 |
| found: | C 41.22 | H 5.61 | Gd 21.98 | N 10.23 |

### f) Octa-Gd complex amide from the Gd complex of 10-(4-carboxy-1-cyclohexyl-2-oxo-3-azabutyl)-1,4,7,10-tetraazacyclododecane-1,4,7-triacetic acid with dendritic octaamine 4d

8.37 g (12 mmol) of the gadolinium complex described in Example 9e and 1.4 g (12 mmol) of N-hydroxysuccinimide are dissolved in 70 ml of DMSO with heating. After cooling to RT, 2.47 g (12 mmol) of N,N′-dicyclohexylcarbodiimide are added, and the mixture is stirred for 60 min. A mixture of 0.67 g (0.5 mmol) of the octaamine described in Example 4d and 3.33 ml (24 mmol) of triethylamine in 70 ml of DMSO is added to the hydroxysuccinimide active ester solution prepared in situ in this way. Stirring at 50° C. overnight is followed by making up the volume to about 1.4 1 with ethyl acetate and stirring for 24 h, and the precipitate is then filtered off with suction, washed with ethyl acetate and dried in vacuo. The residue is dissolved in water and stirred with 2 g of activated carbon for 1 h. The suspension is filtered, the filtrate is ultrafiltered on an AMICON® YM 1 (cut off 1.000 Da), and the retentate is chromatographed with an acetonitrile/water gradient on Lichroprep® RP-18, and the product fractions are freeze dried.

Yield: 1.53 g (42.8%)

Water content (Karl-Fischer): 5.5%

Elemental Analysis (Based on the Anhydrous Substance):

| | | | | |
|---|---|---|---|---|
| calculated: | C 46.45 | H 6.16 | Gd 18.57 | N 11.58 |
| found: | C 46.11 | H 6.37 | Gd 18.10 | N 11.75 |

### EXAMPLE 10

### a) Biphenyl-4-carboxylic acid (2-aminoethyl)amide

2.16 g (9.98 mmol) of biphenyl-4-carbonyl chloride (Aldrich), dissolved in 50 ml of dichloromethane, are added to 60 g (998 mmol) of ethylenediamine in 600 ml of dichloromethane at 0° C., and the mixture is stirred with ice-bath cooling for 2 h and at RT overnight. Subsequently, 300 ml of water are added, and the phases are separated. The organic phase is washed with saturated sodium chloride solution, the aqueous phase is extracted with dichloromethane, and the combined organic phases are dried over sodium sulphate, filtered and concentrated.

Yield: 1.61 g (67.2% of theory)

Elemental Analysis:

| | | | |
|---|---|---|---|
| calculated: | C 74.97 | H 6.71 | N 11.66 |
| found: | C 74.53 | H 6.88 | N 11.90 |

### b) Carboxamide from biphenyl-4-carboxylic acid (2-aminoethyl)amide and Boc-protected G3-(carboxylic acid)dendron

84.1 mg (0.35 mmol) of the biphenyl-4-carboxylic acid (2-aminoethyl)amide described in Example 10a are dissolved in 10 ml of DMF, and 963 mg (0.35 mmol) of the Boc-protected G3-(carboxylic acid)-dendron described in Chemistry—A European Journal 7, 686, (2001) are added. Addition of 0.48 ml (2.8 mmol) of N,N-ethyldiisopropylamine and 208 mg (0.4 mmol) of PyBOP is followed by stirring at RT for 2 days and concentration, and the residue is partitioned between ethyl acetate and sodium bicarbonate solution. The organic phase is washed with water, dried over sodium sulphate, filtered and concentrated.

Yield: 740 mg (71.1% of theory)

Elemental Analysis:

| | | | |
|---|---|---|---|
| calculated: | C 58.94 | H 6.50 | N 14.12 |
| found: | C 58.61 | H 6.83 | N 13.94 |

c) Deprotected Octaamine Dendrimer from 10b

893 mg (0.3 mmol) of the completely protected amine described in Example 10b are dissolved in 15 ml of trifluoroacetic acid, stirred at RT for 1 h and then mixed with 250 ml of diethyl ether, filtered off with suction and thoroughly washed with diethyl ether. The residue is dissolved in water and passed over 75 ml of AMBERLITE® ion exchanger IRA 410 (OH—), and the alkaline eluate is frozen and freeze dried. The resulting colourless powder is employed in the following reaction without further characterization.

Yield: 540 mg

### d) Octa-DTPA Derivative of the Dendrimer Amine 10c

435 mg (0.2 mmol) of the octaamine described in Example 10c are dissolved in 50 ml of water. Then 1.94 g (4.8 mmol) of DTPA monoanhydridemonoethyl ester (Example 13 a of EP 0 331 616) are added in portions in solid form, keeping the pH of the solution at pH 8-9 by adding 2 N sodium hydroxide solution. The mixture is then stirred at this pH and at RT for 1 h, after which the pH is adjusted to 12 by adding further sodium hydroxide solution. The mixture is stirred for a further 5 h and, after the pH has been adjusted to 7 by adding conc. hydrochloric acid, the solution is ultrafiltered through an AMICON® YM 1 (cut off 1.000 Da), and the retentate is chromatographed with an acetonitrile/water gradient on Lichroprep® RP-18. The product fractions are freeze dried and employed in the following complexation without further characterization.

Yield: 1.0 g

e) Octa-GdDTPA Complex of the Dendritic Ligand 10d

1.0 g (0.2 mmol) of the octa-DTPA described in Example 10d is dissolved in 20 ml of water, mixed with 290 mg (0.8 mmol) of gadolinium oxide and stirred at 80° C. for 30 min. The solution is mixed with activated carbon and filtered, and the filtrate is chromatographed with an acetonitrile/water gradient on Lichroprep® RP-18, and the product fractions are freeze dried.

Yield: 924 mg (65.3%)

Water content (Karl-Fischer): 6.9%

Elemental Analysis (Based on the Anhydrous Substance):

| | | | | | |
|---|---|---|---|---|---|
| calculated: | C 39.75 | H 4.04 | Gd 19.10 | N 11.48 | Na 2.79 |
| found: | C 39.31 | H 4.22 | Gd 18.61 | N 11.79 | Na 2.24 |

### EXAMPLE 11

### a) Carboxamide from biphenyl-4-carboxylic acid (2-aminoethyl)amide and (Boc)₈-[G3]-CO₂H

84.1 mg (0.35 mmol) of the biphenyl-4-carboxylic acid (2-aminoethyl)amide described in Example 10a are dissolved in 10 ml of DMF, and 831 mg (0.35 mmol) of the (Boc)₈-[G3]-CO₂H described in European Journal of Organic Chemistry, 1903, (2001) (by hydrolysing the compound 22 described therein) are added. Addition of 0.48 ml (2.8 mmol) of N,N-ethyldiisopropylamine and 208 mg (0.4 mmol) of PyBOP is followed by stirring at RT for 2 days and concentration, and the residue is partitioned between ethyl acetate and sodium bicarbonate solution. The organic phase is washed with water, dried over sodium sulphate, filtered and concentrated.

Yield: 748 mg (82.3% of theory)

Elemental Analysis:

| | | | |
|---|---|---|---|
| calculated: | C 61.05 | H 6.91 | N 8.63 |
| found: | C 60.77 | H 7.05 | N 8.44 |

### b) Deprotected octaamine dendrimer from 11a

649 mg (0.25 mmol) of the completely protected amine described in Example 11a are dissolved in 15 ml of trifluoroacetic acid, stirred at RT for 1 h and then mixed with 250 ml of diethyl ether, filtered with suction and thoroughly washed with diethyl ether. The residue is dissolved in water and passed over 75 ml of AMBERLITE® ion exchanger IRA 410 (OH—), and the alkaline eluate is frozen and freeze dried. The resulting colourless powder is employed in the following reaction without further characterization.

Yield: 430 mg

### c) Octa-Gd complex amide from the Gd complex of 10-(4-carboxy-1-methyl-2-oxo-3-azabutyl)-1,4,7,10-tetraazacyclododecane-1,4,7-triacetic acid with dendritic octaamine 11b

3.02 g (4.8 mmol) of the gadolinium complex of 10-(4-carboxy-1-methyl-2-oxo-3-azabutyl)-1,4,7,10-tetraazacyclododecane-1,4,7-triacetic acid (Example 1f of EP 0946525) and 0.56 g (4.8 mmol) of N-hydroxysuccinimide are dissolved in 25 ml of dimethyl sulphoxide (DMSO) with heating. After cooling to RT, 1.0 g (4.8 mmol) of N,N′-dicyclohexylcarbodiimide is added, and the mixture is stirred for 60 min. A mixture of 359 mg (0.2 mmol) of the octaamine described in Example 11b and 0.97 g (9.6 mmol) of triethylamine in 50 ml of DMSO is added to the hydroxysuccinimide active ester solution prepared in situ in this way. Stirring at 50° C. overnight is followed by making up the volume to about 0.6 l with ethyl acetate and stirring for 24 h, and the precipitate is then filtered off with suction, washed with ethyl acetate and dried in vacuo. The residue is dissolved in water and stirred with 0.5 g of activated carbon for 1 h. The suspension is filtered, the filtrate is ultrafiltered on an AMICON® YM 1 (cut off 1.000 Da), and the retentate is chromatographed with an acetonitrile/water gradient on Lichroprep® RP-18, and the product fractions are freeze dried

Yield: 963 mg (69.0%)

Water content (Karl-Fischer): 5.0%

Elemental Analysis (Based on the Anhydrous Substance):

| | | | | |
|---|---|---|---|---|
| calculated: | C 43.82 | H 5.09 | Gd 18.97 | N 11.61 |
| found: | C 43.43 | H 5.21 | Gd 18.39 | N 11.42 |

### EXAMPLE 12

Plasma Kinetics of the Compounds 2e and 4e After Intravenous Administration to Rats

The title substances from Examples 2e and 4e, and for comparison the title substance from Example 1 of EP 0 836 485, were administered intravenously to rats in a dose of 50 μmol of total gadolinium/kg of body weight. Blood samples were then taken through a catheter in the common carotid artery at various times (1, 3, 5, 10, 15, 30, 60, 90, 120, 240, 360 min, and 24 h p.i.), and the metal content was determined by atomic emission spectroscopy (ICP-AES) and converted into plasma levels using a conversion factor (0.625). The kinetic data were calculated (software: WinNonlin) from the plasma concentrations (Tab. 1, 2).

TABLE 1
Experimental data on the exemplary substances

| | | | Volume of | Total |
|---|---|---|---|---|
| | Half-life | Half-life | distribution | clearance |
| Compound from | α phase | β phase | Vd ss | [ml/min * |
| Example No. | [min] | [min] | [l/kg] | kg] |
| 2e | 3.8 | 46.0 | 0.17 | 5.9 |
| 4e | 3.3 | 42.0 | 0.20 | 4.5 |
| Example No. 1 | 2.4 | 37.5 | 0.13 | 8.1 |
| from EP 0 836 485 | | | | |

TABLE 2
Plasma levels (as % of the dose) of the exemplary
substances up to 24 hours p.i..

| Time | Example No. 1 | Example | Example |
|---|---|---|---|
| [min p.i.] | from EP 0 836 485 | No. 2e | No. 4e |
| 1 | 75.0% | 75.5% | 65.9% |
| 3 | 39.2% | 53.1% | 44.2% |
| 5 | 25.0% | 36.6% | 30.0% |
| 10 | 6.9% | 16.6% | 17.5% |
| 15 | 4.0% | 9.6% | 13.5% |
| 30 | 1.5% | 4.1% | 8.3% |
| 60 | 1.0% | 2.0% | 4.5% |
| 90 | 0.9% | 1.3% | 2.7% |
| 120 | 0.7% | 0.8% | 1.6% |
| 240 | 0.4% | 0.2% | 0.3% |
| 360 | 0.4% | 0.0% | 0.0% |
| 1440 | 0.0% | 0.0% | 0.0% |

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

In the foregoing and in the examples, all temperatures are set forth uncorrected in degrees Celsius and, all parts and percentages are by weight, unless otherwise indicated.

The entire disclosures of all applications, patents and publications, cited herein and of corresponding German application No. 10 2007 002 726.7, filed Jan. 18, 2007, and U.S. Provisional Application Ser. No. 60/885,497, filed Jan. 18, 2007, are incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. Cascade polymer complexes of the general formula (I):
R-L-A-{X—[Y-(Z-{W—K_{w}}_{z})_{y}]ₓ}ₐ₋₁ (I)
where
R=is an HSA-binding unit,
L=is a linker or a bond,
A=is a nitrogen-containing cascade core of base multiplicity a,
X and Y=are independently of one another a direct linkage or a cascade reproduction unit of reproduction multiplicity x and y, respectively,
Z and W=are independently of one another a direct linkage or a cascade reproduction unit of reproduction multiplicity z and w, respectively,
K=is the residue of a complexing agent,
a=is numbers 2 to 12, and
x, y, z and w=are independently of one another the numbers 1 to 4,
with the proviso that exactly one base multiplicity a of the cascade core A represents exactly one point of linkage to L, and
with the proviso that the cascade polymer complexes comprise in the complexing agent residue K in total at least 4 ions of an element of atomic number 20 to 29, 39, 42 to 44 or 57 to 83 and comprise where appropriate cations of inorganic and/or organic bases, amino acids or amino amides.

2. Compound according to claim 1, characterized in that the following applies to the product of the multiplicities: 4≦(a−1)*x*y*z*w≦64.

3. Compound according to claim 1, characterized in that the following applies to the product of the multiplicities 8≦(a−1)*x*y*z*w≦48.

4. Compound according to claim 1, characterized in that R is selected from:

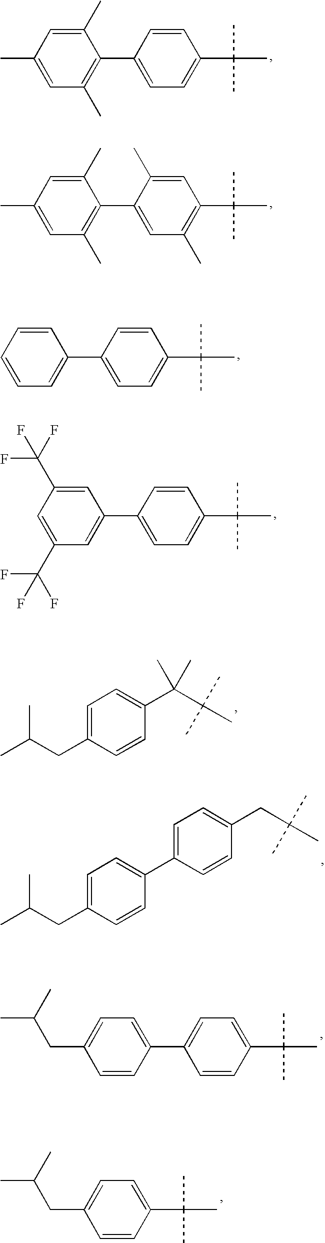

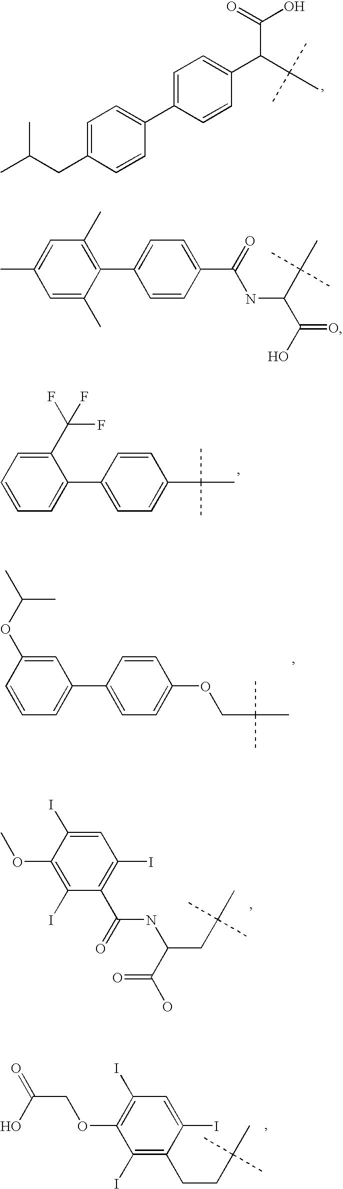

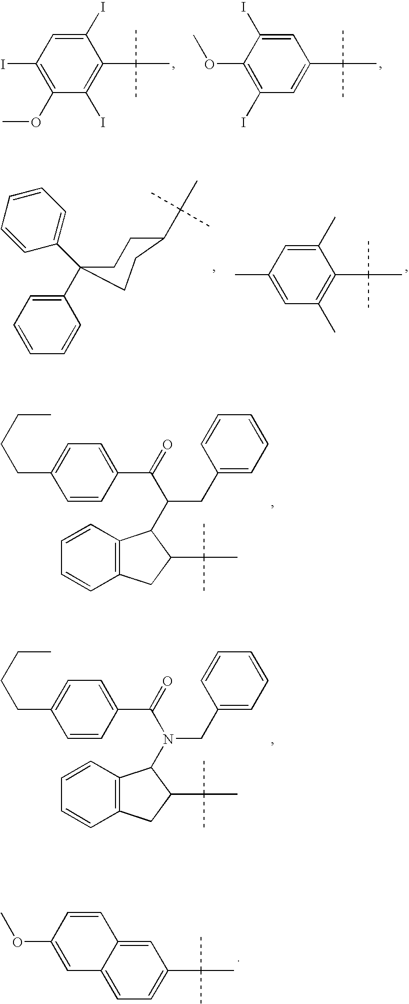

5. Compound according to claim 1, characterized in that L is selected from:
a direct linkage,
—O—CH₂—CO—NH—(CH₂₋CH₂₋O)₁₋₁₀—CH₂—CH₂—CO—,
—O—CH₂—CO—,
—O—CH₂—CO—NH—C₁₋₁₂—CO—,
—CO—,
—OP(O₂)O—C₁₋₁₂—CO—,
—O—CH₂—CO-Pro₄-,
—O—CH₂—CO—NH-aryl-C≡C-aryl-CO—,
—O—CH₂—CO—NH-aryl-C≡C—C≡C-aryl-CO—,
——CO—NH—CH₂—CH₂—,
where Pro is the amino acid proline.

6. Compound according to claim 1, characterized in
that A is selected from:
nitrogen atom, 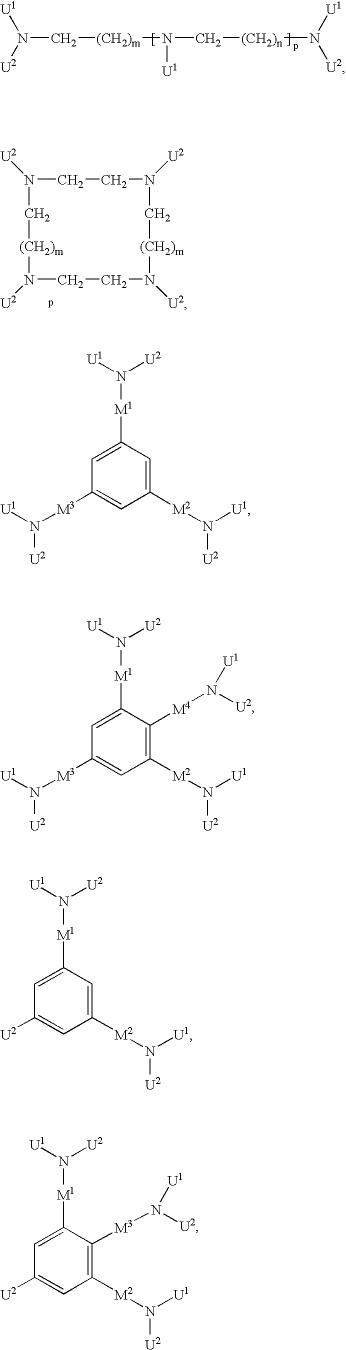 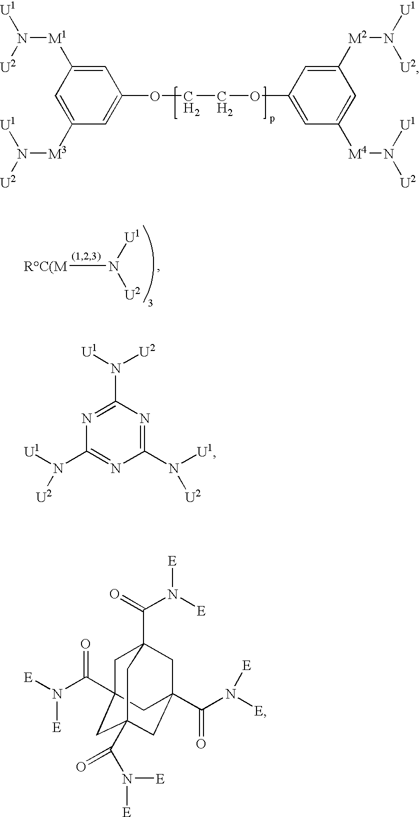
in which
m and n are the numbers 1 to 10,
p is the numbers 0 to 10,
U¹ is Q¹ or E,
U² is Q² or E with
E meaning the group 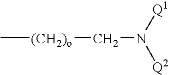
where
o is the numbers 1 to 6,
Q¹ is a hydrogen atom or Q² and
Q² is a direct linkage
M¹, M², M³, M⁴ are independently of one another a direct linkage, a C₁-C₁₀-alkylene chain which is optionally interrupted by 1 to 3 oxygen atoms and/or is optionally substituted by 1 to 2 oxo groups,
M^{(1,2,3)} is meant to denote that in each of the three occurrences of M, M can be selected independently from each other from the common definition for M¹, M², . . . (in other words according to this formula, M can have three times the same meaning or can be different for two or each of the three occurrences),
R^{o} is a branched or unbranched C₁-C₁₀-alkyl radical, a nitro, amino, carboxylic acid group or is 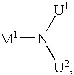
where the number of Q² corresponds to the base multiplicity a, and with the proviso that exactly one Q² represents a linkage to L.

7. Compound according to claim 1, characterized in that A is selected from:
Tris(aminoethyl)amine,
Tris(aminopropyl)amine,
Diethylenetriamine,
Triethylenetetramine,
Tetraethylenepentamine,
1,3,5-Tris(aminomethyl)benzene,
Trimesamide,
Aminoisophthalamide,
3,5-Bis(2-aminoethoxy)benzamide,
3,5-Bis(3-aminopropoxy)benzamide,
3,5-Bis(2-aminoethoxy)aniline,
3,5-Bis(3-aminopropoxy)aniline,
3,4,5-Tris(2-aminoethoxy)benzamide,
3,4,5-Tris(3-aminopropoxy)benzamide,
3,4,5-Tris(2-aminoethoxy)aniline,
3,4,5-Tris(3-aminopropoxy)aniline,
3,5-Diamino-1-benzamide,
1,4,7-Triazacyclononane,
1,4,7,10-Tetraazacyclododecane,
1,4,7,10,13-Pentaazacyclopentadecane,
1,4,8,11-Tetraazacyclotetradecane,
1,4,7,10,13,16-Hexaazacyclooctadecane,
1,4,7,10,13,16,19,22,25,28-Decaazacyclotriacontane,
Tetrakis(aminomethyl)methane,
1,1,1-Tris(aminomethyl)ethane,
Tris(aminopropyl)nitromethane,
2,4,6-Triamino-1,3,5-triazine,
Lysinamide,
Ornithinamide,
Glutamamide,
Aspartamide,
Diaminopropanoamide.

8. Compound according to claim 1, characterized in that the cascade reproduction units X, Y, Z and W are selected independently of one another from: 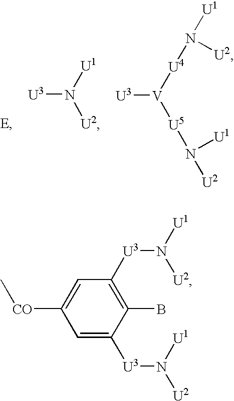
in which
U¹ is Q¹ or E,
U² is Q² or E with
E meaning the group 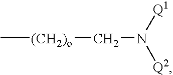
where
o is the numbers 1 to 6,
Q¹ is a hydrogen atom or Q²,
Q² is a direct linkage,
U³ is a C₁-C₂₀-alkylene chain which is optionally interrupted by 1 to 10 oxygen atoms and/or 1 to 2 —N(CO)_{q}—R²—, 1 to 2 phenylene and/or 1 to 2 phenyleneoxy radicals, and/or is optionally substituted by 1 to 2 oxo, thioxo, carboxy, C₁-C₅-alkylcarboxy, C₁-C₅-alkoxy, hydroxy, C₁-C₅-alkyl groups,
where
q is the numbers 0 or 1, and
R² is a hydrogen atom, a methyl or an ethyl radical, which is optionally substituted by 1-2 hydroxy or 1 carboxy group(s),
B is a hydrogen atom or the group 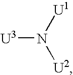
V is the methine group 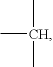
when at the same time U⁴ is a direct linkage or the group M, and U⁵ has one of the meanings of U³, or
V is one of the following groups 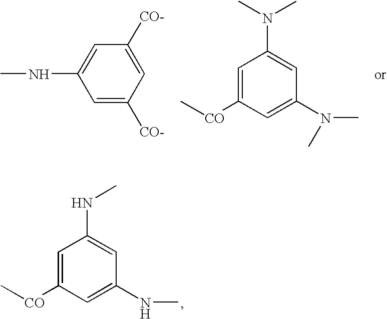
when at the same time U⁴ and U⁵ are identical and are the direct linkage or the group M,
where M is a C₁-C₁₀-alkylene chain which is optionally interrupted by 1 to 3 oxygen atoms and/or is optionally substituted by 1 to 2 oxo groups.

9. Compound according to claim 1, characterized in that the cascade reproduction units X, Y, Z and W are selected independently of one another from:
—CH₂CH₂NH—; —CH₂CH₂N<;
—CO—(CH₂)₂—NH—; —CO—(CH₂)₃—NH—; —CO—(CH₂)₄—NH—; —CO—(CH₂)₅—NH—;
—CO—(CH₂)₆—NH—;
—CO—(CH₂)₂—N<; —CO—(CH₂)₃—N<; —CO—(CH₂)₄—N<; —CO—(CH₂)₅—N<; —CO—(CH₂)₆—N<;
—COCH(NH—)(CH₂)₄NH—; —COCH(N<)(CH₂)₄N<;
—COCH₂OCH₂CON(CH₂CH₂NH—)₂; —COCH₂OCH₂CON(CH₂CH₂N<)₂;
—COCH₂N(CH₂CH₂NH—)₂; —COCH₂N(CH₂CH₂N<)₂;
—COCH₂NH—; —COCH₂N<;
—COCH₂CH₂CON(CH₂CH₂NH—)₂; —COCH₂CH₂CON(CH₂CH₂N<)₂;
—COCH₂OCH₂CONH—C₆H₄—CH[CH₂CON(CH₂CH₂NH—)₂]₂;
—COCH₂OCH₂CONH—C₆H₄—CH[CH₂CON(CH₂CH₂N<)₂]₂;
—COCH₂CH₂CO—NH—C₆H₄—CH[CH₂CON(CH₂CH₂NH—)₂]₂;
—COCH₂CH₂CO—NH—C₆H₄—CH[CH₂CON(CH₂CH₂N<)₂]₂;
—CONH—C₆H₄—CH[CH₂CON(CH₂CH₂NH—)₂]₂;
—CONH—C₆H₄—CH[CH₂CON(CH₂CH₂N<)₂]₂;
—COCH(NH—)CH(COOH)NH—; —COCH(N<)CH(COOH)N<;

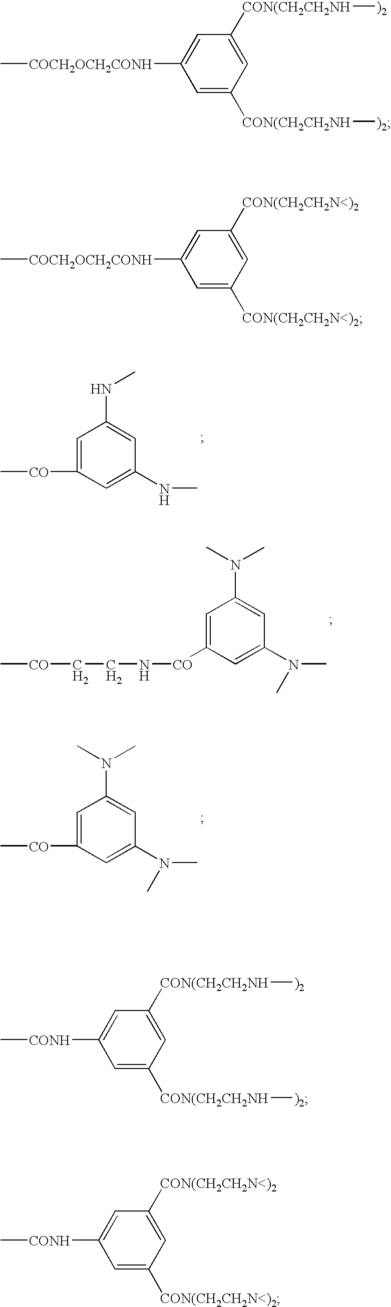

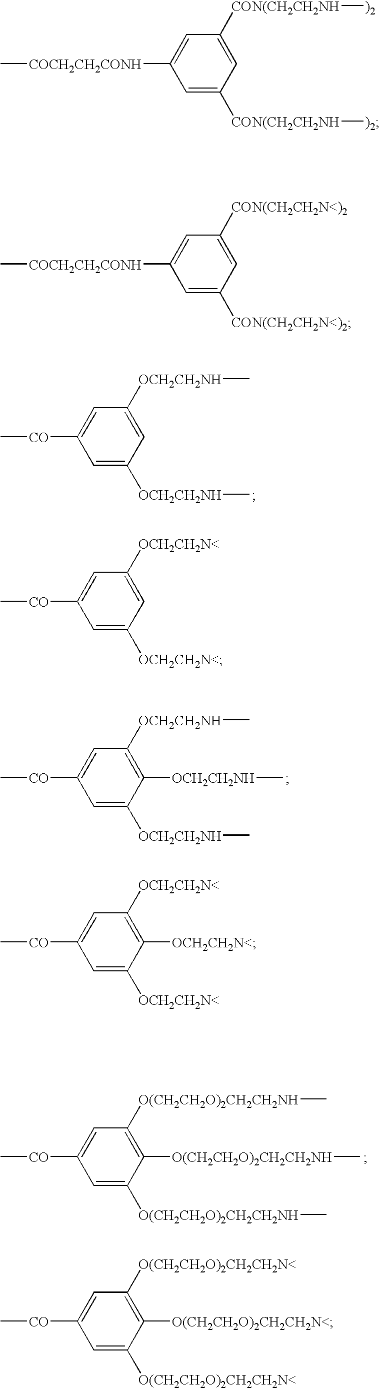

10. Compound according to claim 1, characterized in that the complexing agent residue K is selected according to formula IA, IB or IC: 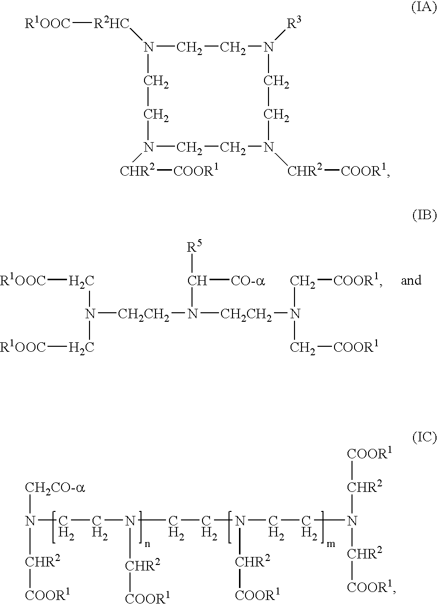
in which
n and m are each the numbers 0, 1, 2, 3 or 4, and where the total of n plus m is not larger than 4,
R¹ are independently of one another a hydrogen atom or a metal ion equivalent of atomic numbers 20-29, 39, 42-44 or 57-83,
R² is a hydrogen atom, a methyl or an ethyl radical, which is optionally substituted by 1-2 hydroxy or 1 carboxy group(s),
R³ is a 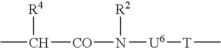
group or a 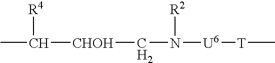
R⁴ is isopropyl, cyclohexyl, a straight-chain, branched, saturated or unsaturated C₁-C₃₀-alkyl chain which is optionally interrupted by 1-10 oxygen atoms, 1 phenylene, 1 phenyleneoxy groups and/or is optionally substituted by 1-5 hydroxy, 1-3 carboxy, 1 phenyl group(s),
R⁵ is a hydrogen atom or is R⁴,
U⁶ is a straight-chain, branched, saturated or unsaturated C₁-C₂₀-alkylene group which optionally comprises 1-5 imino, 1-3 phenylene, 1-3 phenyleneoxy, 1-3 phenyleneimino, 1-5 amide, 1-2 hydrazide, 1-5 carbonyl, 1-5 ethyleneoxy, 1 urea, 1 thiourea, 1-2 carboxyalkylimino, 1-2 ester groups, 1-10 oxygen, 1-5 sulphur and/or 1-5 nitrogen atom(s) and/or is optionally substituted by 1-5 hydroxy, 1-2 mercapto, 1-5 oxo, 1-5 thioxo, 1-3 carboxy, 1-5 carboxyalkyl, 1-5 ester and/or 1-3 amino group(s), where the phenylene groups which are optionally present may be substituted by 1-2 carboxy, 1-2 sulphone or 1-2 hydroxy groups,
T is a —CO-α, —NHCO-α or —NHCS-α group and
α is the point of linkage to the terminal nitrogen atoms of the last generation of the reproduction unit W.

11. Compound according to claim 1, characterized in that the radical R4 in the definition of the complexing agent K according to formula IA or IB is selected from:
isopropyl, cyclohexyl, —CH₃, —C₆H₅, —CH₂—COOH,
—CH₂—C₆H₅, —CH₂—O—(CH₂CH₂—O—)₆CH₃, —CH₂—OH.

12. Pharmaceutical composition comprising a compound according claim 1, where appropriate with the additions customary in pharmaceutical technology.

13. A method for NMR diagnosis comprising administering a compound of claim 1.

14. Process for preparing a pharmaceutical composition, characterized in that the compound according to claim 1 which is dissolved or suspended in water or physiological saline is brought, where appropriate with the additions customary in pharmaceutical technology, into a form suitable for enteral or parenteral administration.
